SEQUENCE LISTING (32 sequences)

SEQ ID NO: 1 - protein, 37 aa, Artificial
  synthetic construct - GLP-1 (1-37)
HDEFERHAEGTFTSDVSSYLEGQAAKEFIAWLVKGRG

SEQ ID NO: 2 - protein, 36 aa, Artificial
  synthetic construct - GLP-1 (1-36)-NH2
HDEFERHAEGTFTSDVSSYLEGQAAKEFIAWLVKGR

SEQ ID NO: 3 - protein, 31 aa, Artificial
  synthetic construct GLP-1 (7-37)
HAEGTFTSDVSSYLEGQAAKEFIAWLVKGRG

SEQ ID NO: 4 - protein, 30 aa, Artificial
  synthetic construct - GLP- 1 (7-36)-NH2
HAEGTFTSDVSSYLEGQAAKEFIAWLVKGR

SEQ ID NO: 5 - protein, 30 aa, Artificial; identical to SEQ ID NO: 4
  synthetic construct - GLP-1 (7-36)

SEQ ID NO: 6 - protein, 29 aa, Artificial
  synthetic construct - GLP-1 (7-35)
HAEGTFTSDVSSYLEGQAAKEFIAWLVKG

SEQ ID NO: 7 - protein, 31 aa, Artificial
  synthetic construct (preferred GLP-1 analog)
XXEGTFTSDVSSYLXGQAAKXFIAWLVKGRG

SEQ ID NO: 8 - protein, 29 aa, Artificial
  synthetic construct (preferred GLP-1 analog)
HAEGTFTSDVSSYLEGQAAKEFIAWLVKX

SEQ ID NO: 9 - protein, 28 aa, Artificial
  synthetic construct - GLP-1 (7-34)
HAEGTFTSDVSSYLEGQAAKEFIAWLVK

SEQ ID NO: 10 - protein, 31 aa, Artificial
  synthetic construct - Val8 GLP (7-37)-OH
HVEGTFTSDVSSYLEGQAAKEFIAWLVKGRG

SEQ ID NO: 11 - protein, 31 aa, Artificial
  synthetic construct - Gln9-GLP-1 (7-37)
HAQGTFTSDVSSYLEGQAAKEFIAWLVKGRG

SEQ ID NO: 12 - protein, 31 aa, Artificial; identical to SEQ ID NO: 11
  synthetic construct - D-Gln9 GLP-1 (7-37)

SEQ ID NO: 13 - protein, 31 aa, Artificial
  synthetic construct - Lys18-GLP-1 (7-37)
HAEGTFTSDVSKYLEGQAAKEFIAWLVKGRG

SEQ ID NO: 14 - protein, 31 aa, Artificial
  synthetic construct - Thr16-Lys18-GLP-1 (7-37)
HAEGTFTSDTSKYLEGQAAKEFIAWLVKGRG

SEQ ID NO: 15 - protein, 30 aa, artificial
  synthetic construct - Gly8-GLP-1 (7-36)-NH2
HGEGTFTSDVSSYLEGQAAKEFIAWLVKGR

SEQ ID NO: 16 - protein, 31 aa, Artificial
  synthetic construct - Acetyl Lys9-GLP-1 (7-37)
HAKGTFTSDVSSYLEGQAAKEFIAWLVKGRG

SEQ ID NO: 17 - protein, 31 aa, artificial
  Synthetic construct - Thr9-GLP-1 (7-37)
HATGTFTSDVSSYLEGQAAKEFIAWLVKGRG

SEQ ID NO: 18 - protein, 31 aa, artificial; identical to SEQ ID NO: 17
  synthetic contruct - D-Thr9-GLP-1 (7-37)

SEQ ID NO: 19 - protein, 31 aa, artificial
  synthetic construct - Asn9-GLP-1 (7-37)
HANGTFTSDVSSYLEGQAAKEFIAWLVKGRG

SEQ ID NO: 20 - protein, 31 aa, artificial; identical to SEQ ID NO: 19
  synthetic construct - D-Asn9-GLP-1 (7-37)

SEQ ID NO: 21 - protein, 31 aa, artificial
  synthetic construct - Ser22-Arg23-Arg24- Gln26-GLP-1 (7-37)
HAEGTFTSDVSSYLESRRAQEFIAWLVKGRG

SEQ ID NO: 22 - protein, 31 aa, artificial
  synthetic construct - Arg23-GLP-1 (7-37)
HAEGTFTSDVSSYLEGRAAKEFIAWLVKGRG

SEQ ID NO: 23 - protein, 31 aa, artificial
  synthetic contruct - Arg24-GLP-1 (7-37)
HAEGTFTSDVSSYLEGQRAKEFIAWLVKGRG

SEQ ID NO: 24 - protein, 31 aa, artificial
  synthetic construct - Met8-GLP-1 (7-37)-OH
HMEGTFTSDVSSYLEGQAAKEFIAWLVKGRG

SEQ ID NO: 25 - protein, 30 aa, artificial; identical to SEQ ID NO: 4
  synthetic construct - a-methyl- Ala8-GLP-1 (7-36)-NH2

SEQ ID NO: 26 - protein, 31 aa, Artificial
  synthetic construct - Gly8-Gln21-GLP-1 (7-37)-OH
HGEGTFTSDVSSYLQGQAAKEFIAWLVKGRG

SEQ ID NO: 27 - protein, 31 aa, artificial
  synthetic construct - LY315902 - His7-IP7- Lys34-C8 Arg26-GLP-1 (7-37)-OH
HAEGTFTSDVSSYLEGQAAREFIAWRVKGRG

SEQ ID NO: 28 - protein, 39 aa, artificial sequence
  synthetic construct - Exendin-4
HGEGTFTSDLSKQMEEEAVRLFIEWLKNGGPSSGAPPPS

SEQ ID NO: 29 - protein, 31 aa, artificial; identical to SEQ ID NO: 3
  synthtic construct - a8-des Arg36-GLP-1 (7-37)

SEQ ID NO: 30 - protein, 31 aa, Artificial
  synthetic construct - liraglutide (Lys26-(N-epsilon-(gamma-Glu(N-alfa-hexadecanoyl)))-Arg34-GLP-1 (7-37)
HAEGTFTSDVSSYLEGQAAKEFIAWLVRGRG

SEQ ID NO: 31 - protein, 31 aa, artificial
  synthetic construct - CJC1131 Lys37(2-(2-(2-maleimideopropionamido(ethoxy)ethoxy) acetamide) GLP-1 (7-37)
HAEGTFTSDVSSYLEGQAAKEFIAWLVKGRK

SEQ ID NO: 32 - protein, 44 aa, artificial
  synthetic construct - ZP10
HGEGTFTSDLSKQMEEEAVRLFIEWLKNGGPSSGAPPSKKKKKK

(19) United States
(12) Patent Application Publication
(10) Pub. No.: US 2009/0181887 A1
(43) Pub. Date: Jul. 16, 2009

(54) USE OF A GLP-1 MOLECULE FOR TREATMENT OF BILIARY DYSKINESIA AND/OR BILIARY PAIN/DISCOMFORT

(75) Inventors: Christian Hansen, Vedbaek (DK); Henrik Nilsson, Copenhagen (DK); Hans T. Schambye, Holte (DK); Tina Geritz Nielsen, Frederiksberg (DK)

Correspondence Address: BROWDY AND NEIMARK, P.L.L.C.;624 NINTH STREET, NW, SUITE 300, WASHINGTON, DC, 20001-5303, US

(73) Assignee: Gastrotech Pharma A/S, Copenhagen (DK)

(21) Appl. No.: 12/066,178

(22) PCT Filed: Sep. 6, 2006
(86) PCT No.: PCT/DK06/00496

§ 371 Date: Mar. 7, 2008

(30) Foreign Application Priority Data
Sep. 8, 2005 (DK) PA200501253

(51) Int. Cl. A61K 38/22; A61P 25/00; G01N 33/566

(52) U.S. Cl. 514/12; 436/86

(57) ABSTRACT

The present invention relates to molecules, compositions and methods suitable for the treatment or prevention of biliary dyskinesia and/or pain and/or discomfort originating from the biliary tree. The peptide hormone glucagon-like peptide-1 (GLP-1) has both anti-secretory effects and smooth muscle relaxatory properties in the gastrointestinal tract. GLP-1 exists in several forms, where the natural occurring GLP-1 molecules are GLP-1 (1-37), GLP-1 (7-37), and the amidated versions GLP-1 (1-36)amide, GLP-1 (7-36)amide. Other molecules capable of binding to and activating the GLP-1 receptor is herein included in the tern GLP-1 molecules. GLP-1 molecules may be naturally occurring or homologues of GLP-1 having one or more amino acid substitutions or modifications. The GLP-1 molecules are used for the manufacture of a medicament for the treatment or prevention of biliary dyskinesia and/or pain and/or discomfort originating from the biliary tree.

All patent and non-patent references cited in the present application, are also hereby incorporated by reference in their entirety.

### FIELD OF INVENTION

The present invention provides molecules, compositions and methods suitable for the treatment or prevention of biliary dyskinesia and/or pain and/or discomfort originating from the biliary tree.

### BACKGROUND OF INVENTION GLP-1

A number of gastrointestinal peptide hormones have both anti-secretory effects and smooth muscle relaxatory properties in the gastrointestinal tract. An especially potent and thereby interesting peptide hormone of this category is glucagon-like peptide-1 (GLP-1). The tissue distribution of GLP-1 has been investigated, with GLP-1 mRNA being detected in rat lung, pancreatic islets, stomach, kidney, hypothalamus and heart but not adipose, liver and skeletal muscle (Bullock et al., “Tissue distribution of messenger ribonucleic acid encoding the rat glucagon-like peptide-1 receptor”, Endocrinology. 1996 July; 137(7):2968-78).

Human GLP-1 (1-37) has the sequence His Asp Glu Phe Glu Arg His Ala Glu Gly Thr Phe Thr Ser Asp Val Ser Ser Tyr Leu Glu Gly Gln Ala Ala Lys Glu Phe Ile Ala Trp Leu Val Lys Gly Arg Gly (SEQ ID NO:1). GLP-1 (1-37) is amidated post-translationally to yield GLP-1 (1-36)NH2, which has the sequence His Asp Glu Phe Glu Arg His Ala Glu Gly Thr Phe Thr Ser Asp Val Ser Ser Tyr Leu Glu Gly Gln Ala Ala Lys Glu Phe Ile Ala Trp Leu Val Lys Gly Arg (NH2) (SEQ ID NO:2); or is enzymatically processed to yield GLP-1 (7-37), which has the sequence His Ala Glu Gly Thr Phe Thr Ser Asp Val Ser Ser Tyr Leu Glu Gly Gln Ala Ala Lys Glu Phe Ile Ala Trp Leu Val Lys Gly Arg Gly (SEQ ID NO:3). GLP-1 (7-37) can also be amidated to yield GLP-1 (7-36)amide, which together with GLP-1 (7-37) constitute the natural forms of the GLP-1 molecule, and which has the sequence His Ala Glu Gly Thr Phe Thr Ser Asp Val Ser Ser Tyr Leu Glu Gly Gln Ala Ala Lys Glu Phe Ile Ala Trp Leu Val Lys Gly Arg (NH₂) (SEQ ID NO:4). Likewise, GLP-1 (1-36)(NH₂) can be processed to GLP-1 (7-36)(NH₂).

The majority of circulating biologically active GLP-1 is found in the GLP-1 (7-36)amide form), with lesser amounts of the bioactive GLP-1 (7-37) form also detectable (Diabetes. 1994 April; 43(4):535-9). Both peptides appear equipotent in all biological paradigms studied to date (Diabetes. 1993 May; 42(5):658-61). GLP-1 is secreted from gut endocrine cells in response to nutrient ingestion and plays multiple roles in metabolic homeostasis following nutrient absorption. An important locus for regulation of GLP-1 biological activity is the N-terminal degradation of the peptide by Dipeptidyl Peptidase (DPP-IV)-mediated cleavage at the position 2 alanine. Strategies designed to circumvent DPP-IV-mediated inactivation of GLP-1 include the generation of modified GLP-1 molecules engineered to exhibit DPP-IV-resistance. Several reports have documented the enhanced biological activity of such molecules in both normal and diabetic rodents. The lizard GLP-1-related peptide exendin-4 has a glycine in position 2, rendering the peptide resistant to DPP-IV-mediated degradation. This likely contributes significantly to the enhanced potency and stability of exendin-4 in vivo. (Diabetes. 1999 May; 48(5):1026-34).

The biological activities of GLP-1 include stimulation of glucose-dependent insulin secretion and insulin biosynthesis, inhibition of glucagon secretion and gastric emptying, and inhibition of food intake. GLP-1 appears to have a number of additional effects in the GI tract and central nervous system (Diabetes 1998 47(2):159-69, Endocrinology. 2001 February; 142(2):521-7, Curr Pharm Des. 2001 September; 7(14):1399-412, Gastroenterology. 2002 February; 122(2):531-44). One effect of GLP-1 is to relax smooth muscle, causing for example slowing of gastric emptying by 50% in man (Wettergren et al, 1993; Gutniak et al, 1996) and inhibition of intestinal motility (Naslund et al., “Glucagon-like peptide-1 analogue LY315902: effect on intestinal motility and release of insulin and somatostatin”, Regul Pept. 2002 Jun. 15; 106(1-3):89-95). Pharmacological levels of GLP-1 appear to inhibit both gastric emptying and gastric acid secretion in humans (Dig Dis Sci. 1993 April; 38(4):665-73)

In studies using rat parietal cell preparations, both exendin-4 and GLP-1 display similar properties with respect to H+ and cAMP production and the actions of these peptides are blocked by the GLP-1 receptor antagonist exendin(9-39) (Eur J Pharmacol. 1994 Oct. 14; 269(2):183-91

### The Biliary System

The biliary system (also called the “biliary tract” or “biliary tree”) consists of the organs and ducts that are involved in the production and transportation of bile (including the bile ducts, gallbladder, cystic duct and the “Sphincter of Oddi” (SO), located where the common bile duct opens into the duodenum), the sphincter being formed by the terminal parts of the common bile duct and pancreatic duct, the common channel and the major duodenal papilla of Vater. The sphincter of Oddi is a smooth muscle structure approximately 1 cm in length that is situated at the junction of the bile duct, pancreatic duct, and duodenum. The two most motile areas of the biliary tract are the gallbladder and the sphincter of Oddi. Furthermore, the bile duct wall is fibromuscular, with smooth muscle cells generally scattered throughout the wall, causing a tonic pressure. The cystic duct can furthermore function as a sphincter by alteration of resistance to flow, and is postulated to be involved in some motor function disorders of the biliary tree (Toouli et al., “Biliary tract motor dysfunction”, Bailliere's Clinical Gastroenterology, Vol. 5, No. 2, June 1991).

### Diseases of the Biliary System

A number of diseases are associated with pain and/or discomfort and/or dyskinesia in the biliary tract (or “biliary dyskinesia”). “Dyskinesia” in the biliary tract is herein defined as abnormal motility of any part of the biliary tract, such as e.g. abnormal motility in the gall bladder, bile duct wall, cystic duct, SO or a sphincter in the gall bladder.

### Defects in Gallbladder Motility

Gallbladder motility disorders are called gallbladder dyskinesia, and the patients who present with this condition have symptoms that are suggestive of gallstones. However, investigations do not reveal evidence of stones. Abdominal pain and/or discomfort is the most common symptom associated with a motility disorder of the gallbladder. The pain is epigastric or in the right upper quadrant. It occurs in episodes and is severe, often lasting for 2 to 3 hours or until it is relieved by analgesics. It may radiate to the back and under the tip of the right scapula. The pain may follow a fatty meal, and it may be associated with nausea and vomiting.

An objective diagnosis of gallbladder dyskinesia is made by evaluating the ability of the gallbladder to empty following a standard stimulus. ^{99m}Tc-labeled iminodiacetic acid is used to study the gallbladder, using a gamma camera and computer analysis. The gallbladder ejection fraction (GBEF) can be estimated by use of the following formula: GBEF (%) C Change in GB activity/Baseline GB activity X 100. It has been shown that the normal gallbladder empties in excess of 50% of its volume in response to a standard meal or a 45-minute intravenous infusion of cholecystokinin octopeptide (CCK-OP; 20 ng/kg/h). In a patient with biliary-type pain, if the GBEF is lower than 40%, this is considered to be abnormal. Once gallbladder dyskinesia has been diagnosed, there are few treatment options other than cholecystectomy or endoscopic sphincterotomy, because current pharmacotherapy does not provide relief.

A defect of gallbladder motility is a significant factor in cholesterol gallstone formation (Portincasa, P et al. “In vitro studies of gall-bladder smooth muscle function: relevance in cholesterol gallstone disease”, Ailment Pharmacol Ther 2000, 14(suppl 2):19-26).

Data suggest that gall-bladder motility in the interdigestive (i.e. fasting state) state may have an important role in gallstone pathogenesis (van Erpecum K J et al. “Gallstones: an intestinal disease?”) Impaired bile movement will prolong the residence of bile in the gall bladder, thus allowing more time for nucleation of cholesterol crystals from the supersaturated bile. Gallbladder motility is also linked to inflammatory bowel disease (Masclee et al. Dig Liver Dis. 2003 July; 25 Suppl 3:S35-8).

Patients at high risk of gallstone disease, and patients who are treated chronically with gall-bladder motility inhibiting drugs, would benefit from improved gall-motility using a prokinetic agent, such as the agents disclosed herein.

Furthermore, presence of pain associated with abnormal gallbladder motility has been reported in different clinical situations such as chronic acalculous cholecystitis, biliary dyskinesia, cystic duct syndrome, alcalculous gallbladder disease (characterized by defective gallbladder emptying) and postcholecystectomy syndrome.

Pain associated with acalculous gallbladder disease is thought to be due to disordered gallbladder motility. Patients on long-term parenteral nutrition have been shown to have large volume gallbladders which demonstrate sluggish contraction. Sludge often forms in the gallbladders of these patients and this may lead to gallstone formation.

Gallbladder motility may also be deleteriously affected by obesity, diabetes mellitus or coeliac disease (Fraquelli et al., Dig Liver Dis. 2003 July; Suppl 3:S12-6). Further studies have shown a link between diabetic neuropathy and gall bladder contractility (Geller et al., Probl Endokrinol (Mosk.) 1991 May-June; 37(3):8-10).

Gallbladder hypomotility is also associated with Downs syndrome, and probably causes the high prevalence of gallstones in this patient group (Tasdemir et al., J Pediatr Gastronterol Nutr. 2004 August; 39(2):187-91).

Beta-thalassemia patients have gall-bladder dysmotility, which contributes to the pathogenesis of pigment gallstones/sludge (Portincasa et al., World J Gastroenterol 2004; 10(16):2383-2390).

Gall bladder motility is often impaired in high risk situations for gallstone formation, such as pregnancy, obesity, diabetes mellitus, gastric surgery, very low calorie dieting and total parenteral nutrition, as well as extracorporeal shockwave lithotripsy (Pauletzki J et al., “Gallbladder emptying and gallstone formation: a propective study on gallstone recurrence” Gastroenterology 1996; 111:765-71). Hyperthyroidism is associated with increased incidence of gallstones, due to causing a decrease in biliary motility. This is probably due to hyperthyroidism causing increased tension of the sphincter of Oddi, preventing normal bile flow and increasing the probability of common bile duct stones (Inkinen j et al., “Direct effect of thyroxine on pig sphincter of Oddi contractility”, Dig Dis Sol 2001, 46:182-186). Somatostatin-producing tumours also induce impaired gallbladder motility.

### SO Dysfunction

The term “Sphincter of Oddi dysfunction” is used to describe motility abnormalities caused by either “sphincter of Oddi stenosis” or “sphincter of Oddi dyskinesia”. Both conditions are associated with obstruction to bile flow through the sphincter of Oddi, causing retention of bile in the biliary tree and pancreatic juice in the pancreatic duct. Disorders of the sphincter of Oddi (SO) may take the form of: (1) stenosis from glandular hyperplasia, muscle hyperplasia/hypertrophyor fibrosis; or (2) dyskinesia due to muscular incoordination or muscular hypertonicity (spasm).

Often, patients with SO dysfunction present after cholecystectomy for either calculous or non-calculous gallbladder disease (Bar-Meir, 1984, “Frequency of papillary dysfunction among cholecystectomized patients”, Hepatology 4:328-330).

SO stenosis is associated with possible structural alteration, which is characterised by manometry as an elevated basal SO pressure (above 40 mm Hg) in contrast to SO dyskinesia, which represents a possible smooth muscle dysfunction, SO stenosis may be due to fibrotic changes in the sphincter or to smooth muscle hyperplasia. Various types of smooth muscle dysfunction causing SO dyskinesia have been characterised, including rapid phasic contraction and excessive retrograde contractions. High frequency contractions will produce a transient obstruction to bile flow and reduce the overall bile flow rate: a frequency in excess of seven contractions per minute is considered abnormal.

Secondary damage to the sphincter may result from the passage of small stones or follow inflammation in either the binary tract or the pancreas. These events may result in repair by fibrosis, which may lead to a fixed stenosis reflected manometrically by a high basal pressure.

Biliary-like pain and SO dysfunction may involve abnormalities either in the biliary sphincter, pancreatic sphincter, or both.

Current treatment methods for SO dysfunction are aimed at reducing the resistance caused by the SO to the flow of bile or pancreatic juice: nifedipine, isosorbide dinitrate, injecting the sphincter with botulinum toxin (e.g. from Botox; Allergan, Inc., Irvine, Calif.), endoscopic sphincterectomy or hydrostatic balloon dilation may be considered.

### Factors Inhibiting Billary Tract Motility

Inhibitory factors that reduce the basal tone of the biliary system are well known and cause an increased risk of biliary motility disorders, including an increased risk of gallstone formation.

Various drugs are known to impair the passage of bile, mostly by inhibiting gallbladder emptying, including somatostatin, octreotide, Sandostatin LAR, anticholinergc drugs, Nitric oxide, L-Arginine, nitric oxide donors such as glyceryl trinitrate, nitroprusside, calcium chanel antagonists such as nlifedipine and verapamil, loperamide, Nifedipine, progesterone, Trimebutine maleate, Loperamide, ondansetron (see also van Erpecum et al., “Review article: agents affecting gall-bladder motility—role in treatment and prevention of gallstones”, Aliment Pharmacol ther 2000: 14 (Suppl. 2): 66-70).

Since gallstone formation is associated with impaired gallbladder contraction, chronic use of these drugs may lead to the development of biliary sludge or stones.

### Factors Inducing Bile Flow

Known excitatory factors capable of inducing bile flow may also be used to treat motility disorders, and include: Cholecystokinin (CCK), caerulein, acetylcholine, motilin, cholinomimetic drugs such as bethanecol or prostigmine, ursodeoxcholic acid, dietary fat, NSAIDS, erythromycin, cisapride, cholestyramine, .v. administration of amino acids, and non-steroidal anti-inflammatory drugs (see also van Erpecum et al., “Review article: agents affecting gall-bladder motility—role in treatment and prevention of gallstones”, Aliment Pharmacol ther 2000: 14 (Suppl. 2): 66-70).

### Other Biliary Tract Disorders Resulting in Deleterious Biliary Motility Changes

### Biliary Obstruction

One of the most common causes of extrahepatic binary obstruction is choledocholithiasis with one or more stones in the common bile duct or common hepatic duct causing biliary obstruction. Up to 10% of patients with gallstones have common bile duct stones. Common bile duct stones have been discovered days to several years after surgery in as many as 5% of patients who have undergone cholecystectomy (Am J Surg. 1989; 158:171-173). It is believed the stones represent either retained stones or stones that have formed de novo after the operation. Stones in the bile duct can cause biliary obstruction and cholestasis.

### Cholangiocarcinoma

Cholangiocarcinoma is an adenocarcinoma of the intrahepatic or extrahepatic bile duct. Primary sclerosing cholangitis (PSC) is a major risk factor for the development of cholangiocarcinoma. Either can cause partial or total biliary tract obstruction.

### Other Tumour Types

Although the majority of bile duct tumors are malignant, some benign biliary lesions result in biliary obstruction and cholestasis. These include papillomas, adenomas and cystadenomas (Anthony PP. Tumors of the hepatobiliary system. In: Diagnostic Histopathology of Tumors. Fletcher CDM, Editor. London. Churchill ULvingstone; 2000; 411-460).

Tumors of the ampulla of Vater can be benign (adenomas) or malignant (ampullary carcinoma). Either can result in partial or total biliary tract obstruction.

### Mirizzi's Syndrome

Mirizzi's syndrome is caused by an impacted cystic duct stone leading to gallbladder distention and subsequent compression of the extrahepatic biliary tree. Occasionally the gallstone erodes into the common hepatic duct producing a cholecystocholedochal fistula. The original classification of Mirizzi's syndrome has been expanded to include hepatic duct stenosis caused by a stone at the junction of the cystic and hepatic ducts or as a result of cholecystitis even in the absence of a obstructing cystic duct stone (Hepatogastroenterology. 1997; 44:63-67).

### AIDS Cholangiopathy

Cholestasis can be seen in AIDS as a result of biliary ductal changes seen on cholangiogram resembling primary sclerosing cholangitis. The ductal strictures are thought to be caused by infections including cryptosporidia, cytomegalovirus, microsporidium and Cyclospora (Semin Liver Dis. 1997; 17:335-344). Patients present with right upper quadrant pain.

### Parasites

Extrahepatic partial or complete biliary obstruction has also been seen with various parasitic infections such as Strongyloides, Ascaris, and liver flukes such as Clonorchis sinensis and Fasciola hepatica.

### Further Disorders

Other causes of disorders of the biliary system include, but are not restricted to:
Obstructive lesion in the large ducts of the biliary tree outside the liver.
Primary biliary cirrhosis
Primary sclerosing cholangitis
Infantile obstructive cholangiopathy—e.g. extrahepatic biliary atresia
Damage to the small intraheptic bile ducts by e.g. drugs such as benoxaprofen, chlorpromazine, haloperidol, imipramine etc.
biliary sludge
cholestasis

### Measurement of Biliary System Motility

The motility of the various parts of the biliary system may be measured in individuals by using standard protocols, such as e.g. the protocols described by Portincasa et al., “Standards for diagnosis of gastrointestinal motility disorders”; Dig Liver Dis 2000; 32: 160-172.

Animal models may also be suitable for examining the effect of an agent on the biliary system, such as e.g. the ovine model described by Romanski et al. (Pol J Pharmacol. 2004 March-April; 56(2):247-56).

### SUMMARY OF INVENTION

One aspect of the present invention relates to the use of a GLP-1 molecule for the manufacture of a medicament for the treatment of binary dyskinesia.

In a second aspect, the methods and compositions according to the present invention may be used in the treatment of any type of pain or discomfort originating from the biliary system.

Without being bound by theory, it is contemplated that the GLP-1 molecule works through action on the motility of the biliary tract.

Thus, according to one aspect of the present invention, there is provided the use of a glucagon-like peptide-1 molecule for the manufacture of a pharmaceutical composition for the treatment of pain and/or dyskinesia of the biliary system.

Further according to the invention it may be preferable to combine said GLP-1 molecule with one or more other gastrointestinal peptide hormone(s) or derivative(s) thereof in the pharmaceutical composition in order to achieve complementary effects. In a particularly preferred embodiment PYY1-36, PYY3-36 or a derivative/analogue thereof is combined with a GLP-1 molecule in the pharmaceutical composition. Further, the GLP-1 molecule may be combined with Pancreatic Polypeptide (PP).

According to another aspect of the present invention there is provided a combination, optionally in the form of a pharmaceutical composition, which may be used for the treatment of pain and/or dyskinesia in the binary system, characterized in that it comprises a combination of at least one GLP-1 molecule together with one or more of: analgetics, such as an opioid, an NSAID, an salicylic acid derivative, a paracetamol, or agents impacting biliary dyskinesia such as a nitrate or a calcium channel antagonist.

### DETAILED DESCRIPTION OF THE INVENTION

### Definitions

Affinity: the strength of binding between receptors and their ligands, for example between an antibody and its antigen.

Amino Acid Residue: An amino acid formed upon chemical digestion (hydrolysis) of a polypeptide at its peptide linkages. The amino acid residues described herein are preferably in the “L” isomeric form. However, the amino acid encompasses every amino acid such as L-amino acid, D-amino acid, alpha-amino acid, beta-amino acid, gamma-amino acid, natural amino acid and synthetic amino acid or the like as long as the desired functional property is retained by the polypeptide. Further included are natural or synthetic amino acids which have been modified. NH2 refers to the free amino group present at the amino terminus of a polypeptide. COOH refers to the free carboxy group present at the carboxy terminus of a polypeptide. Standard polypeptide abbreviations for amino acid residues are used herein.

It should be noted that all amino acid residue sequences represented herein by formulae have a left-to-right orientation in the conventional direction of amino terminus to carboxy terminus. Furthermore, it should be noted that a dash at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

Concentration equivalent: A concentration equivalent is an Equivalents dosage being defined as the dosage of a ghrelin-like compound having in vitro and/or in vivo the same response as evaluated from a dosage-response curve of wild-type ghrelin.

Dissociation constant, Kd: a measure to describe the strength of binding (or affinity or avidity) between receptors and their ligands, for example an antibody and its antigen. The smaller Kd the stronger binding.

Biliary dyskinesia: any motility abnormality in the binary tree that causes pain and/or discomfort in the patient. This includes but is not restricted to gallbladder dysfunction, dysfunction of the biliary tract and dysfunction of the Sphincter of Oddi. Biliary dysfunction may e.g. be increased motility of an area of the biliary tract, or decreased motility of an area of the biliary tract, or alternatively disordered control of motility, such as e.g. with spasms in the biliary tract.

Gallbladder dysfunction: any motility abnormality of the gall bladder including abnormal gallbladder emptying that causes biliary-type pain or discomfort.

SO dysfunction is the term used to define motility abnormalities of the sphincter of Oddi (see Rome II: The functional Gastrointestinal Disorders 2^{nd} Edition) Individual: A living animal. In preferred embodiments, the subject is a mammal, including humans and non-human mammals such as dogs, cats, pigs, cows, sheep, goats, horses, rats, and mice. In the most preferred embodiment, the subject is a human.

Isolated: is used to describe the various GLP-1 molecules, polypeptides and nucleotides disclosed herein, that have been identified and separated and/or recovered from a component of its natural environment or of its production process. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified.

Modified amino acid: an amino acid wherein an arbitrary group thereof is chemically modified. In particular, a modified amino acid chemically modified at the alpha-carbon atom in an alpha-amino acid is preferable.

Polypeptide: The phrase polypeptide refers to a molecule comprising amino acid residues which do not contain linkages other than amide linkages between adjacent amino acid residues.

Surfactant molecule: Molecule comprising a hydrophobic part and a hydrophilic part, i.e. molecule capable of being present in the interphase between a lipophilic phase and a hydrophilic phase.

### Indications

One aspect of the present invention relates to use of a GLP-1 molecule in the manufacture of a medicament for the treatment of individuals suffering from, or at risk of suffering from, pain or discomfort and/or dyskinesia in the biliary tract (or “biliary dyskinesia”). Thus, the present invention relates to the treatment of individuals suffering from, or at risk of suffering from, biliary dyskinesia and/or pain or discomfort originating from the biliary tract associated with any of the following pathological conditions:
inflammatory bowel disease
chronic acalculous cholecystitis
alcalculous gallbladder disease
a cystic duct syndrome
acalculous gallbladder disease
postcholecystectomy syndrome
Treatment with gall-bladder motility inhibiting drugs
parenteral nutrition
obesity
diabetes mellitus
coeliac disease
diabetic neuropathy
Downs syndrome
Beta-thalassemia
Pregnancy
gastric surgery
very low calorie dieting
extracorporeal shockwave lithotripsy
Somatostatin-produciong tumour
“sphincter of Oddi stenosis”
“sphincter of Oddi dyskinesia”
glandular hyperplasia, muscle hyperplasia/hypertrophy or fibrosis,
SO muscular incoordination or muscular hypertonicity (spasm)
Hyperthyroidism
Treatment with a compound known to impair the passage of bile, such as somatostatin, octreotide, Sandostatin LAR, anticholinergc drugs, Nitric oxide, L-Arginine, nitric oxide donors such as glyceryl trinitrate, nitroprusside, calcium chanel antagonists such as nifedipine and verapamil, loperamide, Nifedipine, progesterone, Trimebutine maleate, Loperamide, ondansetron
Biliary tract obstruction (partial or complete), such as caused by choledocholithiasis
Cholangiocarcinoma
Primary sclerosing cholangitis (PSC)
Tumors of the ampulla of Vater
Other biliary tumour types, including papillomas, adenomas and cystadenomas
Mirizzi's syndrome
AIDS cholangiopathy
infections including cryptosporidia, cytomegalovirus, microsporidium and Cyclospora.
Parasite infections, such as Strongyloides, Ascaris, and liver flukes such as Clonorchis sinensis and Fasciola hepatica
Obstructive lesion in the large ducts of the biliary tree outside the liver.
Primary biliary cirrhosis
Primary sclerosing cholangitis
Infantile obstructive cholangiopathy—e.g. extrahepatic biliary atresia
Damage to the small intraheptic bile ducts by e.g. drugs such as benoxaprofen, chlorpromazine, haloperidol, imipramine etc.
biliary sludge
cholestasis

In another embodiment, the present invention relates to the treatment of individuals suffering from, or at risk of suffering from, biliary dyskinesia and/or pain or discomfort originating from the biliary tract associated with gallstones.

The pain or discomfort and/or dyskinesia treated using the methods of the present invention may be present in any part(s) of said individual's biliary tract, thus, the present invention may be e.g. used to treat pain and/or discomfort and/or dyskinesia of one or more of the following:
gallbladder and/or
sphincter of Oddi and/or
a bile duct wall and/or
a cystic duct and/or

Thus, in one preferred embodiment, said individual is suffering from, or at risk of suffering from, a pathological condition causing gallbladder dyskinesia and/or pain and/or discomfort. Said individual may for example be suffering from, or at risk of suffering from, any of the following pathological conditions:
biliary dyskinesia
gallstones
inflammatory bowel disease
chronic acalculous cholecystitis
alcalculous gallbladder disease
cystic duct syndrome
acalculous gallbladder disease
postcholecystectomy syndrome

In another embodiment, said individual is suffering from a pathological condition associated with causing gallbladder dyskinesia and/or pain and/or discomfort. Thus, for example, said individual may be suffering from, or at risk of suffering from, gallbladder dyskinesia and/or pain associated with any of the following pathological conditions:
Treatment with gall-bladder motility inhibiting drugs
parenteral nutrition
obesity
diabetes mellitus
coeliac disease
diabetic neuropathy
Downs syndrome
Beta-thalassemia
Pregnancy
gastric surgery
a very low calorie dieting
a extracorporeal shockwave lithotripsy
Somatostatin-producing tumour

In another embodiment, the individual to be treated according to the present invention is suffering from, or at risk of suffering from, a pathological condition causing SO dyskinesia and/or pain and/or discomfort. Thus, for example, said individual may be suffering from, or at risk of suffering from, “sphincter of Oddi stenosis” (caused e.g. by glandular hyperplasia, muscle hyperplasia/hypertrophy or fibrosis), and/or “sphincter of Oddi dyskinesia (caused e.g. by muscular incoordination or muscular hypertonicity (spasm)). In one preferred embodiment of said SO dyskinesia, the individual has an abnormal SO contraction of seven contractions per minute or more. Furthermore, the SO dyskinesia and/or pain and/or discomfort may involve abnormalities in e.g. the biliary sphincter, pancreatic sphincter, or both, In one preferred embodiment of the present invention, the SO dyskinesia may be associated with hyperthyroidism, thus in one embodiment of the present invention, the individual to be treated suffers from, or is at risk of suffering from, hyperthyroidism

In another embodiment of the present invention, the individual to be treated has been, is being or is going to be treated with an inhibitory factor (such as a drug) known to inhibit biliary tract motility and/or reduce the basal tone of the biliary system. Said inhibitory factor may be, but is not restricted to, any of the following factors: somatostatin, octreotide, Sandostatin LAR, anticholinergc drugs, Nitric oxide, L-Arginine, nitric oxide donors such as glyceryl trinitrate, nitroprusside, calcium chanel antagonists such as nifedipine and verapamil, loperamide, Nifedipine, progesterone, Trimebutine maleate, Loperamide or ondansetron.

Other biliary tract disorders causing biliary dyskinesia and/or pain and/or discomfort that may be treated using the methods and compositions of the present invention include, but are not restricted to:
Biliary tract obstruction (partial or complete), such as caused by choledocholithiasis
Cholangiocarcinoma
Primary sclerosing cholangitis (PSC)
Tumors of the ampulla of Vater
Other biliary tumour types, including papillomas, adenomas and cystadenomas
Mirizzi's syndrome
AIDS cholangiopathy
infections including cryptosporidia, cytomegalovirus, microsporidium and Cyclospora.
Parasite infections, such as Strongyloides, Ascaris, and liver flukes such as Clonorchis sinensis and Fasciola hepatica
Obstructive lesion in the large ducts of the biliary tree outside the liver.
Primary biliary cirrhosis
Primary sclerosing cholangitis
Infantile obstructive cholangiopathy—e.g. extrahepatic biliary atresia
Damage to the small intraheptic bile ducts by e.g. drugs such as benoxaprofen, chlorpromazine, haloperidol, imipramine etc.
biliary sludge
cholestasis

### GLP-1 Molecule

The present invention relates to the use of GLP-1 molecule(s) in the manufacture of a medicament for the treatment of biliary tract dyskinesia and/or pain and/or discomfort originating from the biliary system. The term “GLP-1 molecule” is used herein to refer to any molecule capable of binding to and activating the GLP-1 receptor. Methods for assaying the functional activity of the GLP-1 molecules for use in the present invention are described below in the section entitled “Functional activity of GLP-1 molecule”. It is to be understood that the activity of the GLP-1 molecules for use in the present invention can be less potent or more potent than native GLP-1 (7-36)amide.

Preferably, the GLP-1 molecule for use in the present invention is a polypeptide. One preferred group of compounds with this activity are the GLP-1 polypeptides originally sequenced from natural sources. Thus, a preferred molecule for use in the present invention may be selected from the group consisting of: GLP-1 (7-36)amide, and GLP-1 (7-37).

The present invention further includes the use of recombinant or synthetically produced human GLP-1 peptides as well as GLP-1 peptides derived from other species, whether recombinant or synthetic. Thus in one embodiment, the GLP-1 molecule is a homologue of GLP-1.

In one preferred embodiment of the present invention, the GLP-1 molecule is a GLP-1 analog. A “GLP-1 analog” is defined as a molecule having one or more (such as 15 or fewer, for example 13 or fewer, such as 11 or fewer, for example 9 or fewer, such as 7 or fewer, for example 5 or fewer, such as 3 or fewer, for example 2 or fewer, such as 1 or fewer) amino acid substitutions, deletions, inversions, or additions relative to GLP-1 (7-37) and may include the D-amino acid forms. Numerous GLP-1 analogs are known in the art and include, but are not limited to, GLP-1 (7-34), GLP-1 (7-35), GLP-1 (7-36) NH2, Gln9-GLP-1 (7-37), d-Gln9-GLP1 (7-37), Thr16-Lys18-GLP-1 (7-37), and Lys18-GLP-1 (7-37), Gly′-GLP-1 (7-36) NH2, Gly′-GLP1 (7-37) OH, Val′-GLP-1 (7-37) OH, Met8-GLP-1 (7-37) OH, acetyl-Lys9-GLP-1 (7-37), Thr9GLP-1 (7-37), D-Thr9-GLP-1 (7-37), Asn9-GLP-1 (7-37), D-Asn9-GLP-1 (7-37), Ser2-Arg23 Arg24-Gln26-GLP-1 (7-37), Arg23-GLP-1 (7-37), Arg24-GLP-1 (7-37), a-methyl-Ala8-GLP-1 (736) NH2, and Gly′-Gln2′-GLP-1 (7-37) OH, and the like.

Other GLP-1 analogs consistent with the present invention are described by the formula:

RI-X-Glu-Glyl0-Thr-Phe-Thr-Ser-Asp15-Val-Ser-Ser-Tyr-Leu20-Y-Gly-Gln-Ala-Ala25 Lys-Z-Phe-Ile-Ala3-Trp-Leu-Val-Lys-Gly35-Arg-R2 (SEQ ID NO: 7) wherein: R, is selected from the group consisting of L-histidine, D-histidine, desaminohistidine, 2-amino-histidine, beta-hydroxy-histidine, homohistidine, alpha-fluoromethylhistidine, and alpha-methyl-histidine;
X is selected from the group consisting of Ala, Gly, Val, Thr, Ile, and alpha-methyl-Ala;
Y is selected from the group consisting of Glu, Gln, Ala, Thr, Ser, and Gly; Z is selected from the group consisting of Glu, Gln, Ala, Thr, Ser, and Gly; and R2 is selected from the group consisting of NH2, and Gly-OH.

GLP-1 analogs also have been described in WO 91/11457, and include GLP-1 (7-34), GLP-1 (7-35), GLP-1 (7-36), or GLP-1 (7-37), or the amide form thereof, and pharmaceutically-acceptable salts thereof, having at least one modification selected from the group consisting of:

(a) substitution of glycine, serine, cysteine, threonine, asparagine, glutamine, tyrosine, alanine, valine, isoleucine, leucine, methionine, phenylalanine, arginine, or D-lysine for lysine at position 26 and/or position 34; or substitution of glycine, serine, cysteine, threonine, asparagine, glutamine, tyrosine, alanine, valine, isoleucine, leucine, methionine, phenylalanine, lysine, or a D-arginine for arginine at position 36;
(b) substitution of an oxidation-resistant amino acid for tryptophan at position 31;
(c) substitution of at least one of: tyrosine for valine at position 16; lysine for serine at position 18; aspartic acid for glutamic acid at position 21; serine for glycine at position 22; arginine for glutamine at position 23; arginine for alanine at position 24; and glutamine for lysine at position 26; and
(d) substitution of at least one of: glycine, serine, or cysteine for alanine at position 8; aspartic acid, glycine, serine, cysteine, threonine, asparagine, glutamine, tyrosine, alanine, valine, isoleucine, leucine, methionine, or phenylalanine for glutamic acid at position 9; serine, cysteine, threonine, asparagine, glutamine, tyrosine, alanine, valine, isoleucine, leucine, methionine, or phenylalanine for glycine at position 10; and glutamic acid for aspartic acid at position 15; and
(e) substitution of glycine, serine, cysteine, threonine, asparagine, glutamine, tyrosine, alanine, valine, isoleucine, leucine, methionine, or phenylalanine, or the D- or N-acylated or alkylated form of histidine for histidine at position 7; wherein, in the substitutions described in (a), (b), (d), and (e), the substituted amino acids can optionally be in the D-form and the amino acids substituted at position 7 can optionally be in the N-acylated or N-alkylated form.

Preferred GLP-1 molecules used in the present inventive formulation also include analogs of GLP-1 (7-37) NH2 and GLP-1 (7-37) in which one or more amino acids which are not present in the original sequence are added or deleted, and derivatives thereof.

Specifically, His and desamino-histidine are preferred for R and/or Ala, Gly and Val are preferred at the “X” position. Also, Glu and Gln are preferred for at the “Y” position. Glu and Gln are preferred at the “Z” position and Gly-OH is preferred for R2.

A particularly preferred GLP-1 analog is known as Val (8) GLP-1(V8GLP-1) and has a formula according to SEQ ID NO: 7, wherein R, is L-histidine, X is Val, Y is Glu, Z is Glu and R2 is Gly-OH.

In another preferred embodiment of the present invention, the GLP-1 molecule is a GLP-1 derivative. A “GLP-1 derivative” is defined as a molecule having the amino acid sequence of GLP-1 (7-37) or of a GLP-1 analog, but additionally comprises chemical modification of one or more of its amino acid side groups, a-carbon atoms, terminal amino group, or terminal carboxylic acid group. A chemical modification includes, but is not limited to, adding chemical moieties, creating new bonds, and removing chemical moieties. Modifications at amino acid side groups include, without limitation, acylation of lysine s-amino groups, N alkylation of arginine, histidine, or lysine, alkylation of glutamic or aspartic carboxylic acid groups, and deamidution of glutamine or asparagine. Modifications of the terminal amino include, without limitation, the des-amino, N-lower alkyl, N-di-lower alkyl, and N-acyl modifications. Modifications of the terminal carboxy group include, without limitation, the amide, lower alkyl amide, dialkyl amide, and lower alkyl ester modifications. Lower alkyl is C1-C4 alkyl. Furthermore, one or more side groups, or terminal groups, may be protected by protective groups known to the ordinarily-skilled protein chemist The a-carbon of an amino acid may be mono- or dimethylated.

Other GLP-1 derivatives include molecules which are selected from the group consisting of a peptide having the amino acid sequence:

NH2-His′-Ala-Glu-Glyl-Thr-Phe-Thr-Ser-Asp′5-Val-Ser-Ser-Tyr-Leu2-Glu-Gly Gln-Ala-Ala25-Lys-Glu-Phe-Ile-Ala30-Trp-Leu-Val-X (SEQ ID NO; 8) and pharmaceutically-acceptable salts thereof, wherein X is selected from the group consisting of:—
Lys and Lys-Gly; and a derivative of said peptide, wherein said peptide is selected from the group consisting of: a pharmaceutically-acceptable lower alkylester of said peptide; and a pharmaceutically-acceptable amide of said peptide, selected from the group consisting of amide, lower alkyl amide, and lower dialkyl amide.

Yet other GLP-1 derivatives appropriate for use in the present invention include compounds claimed in U.S. Pat. No. 5,512,549 described by the formula:

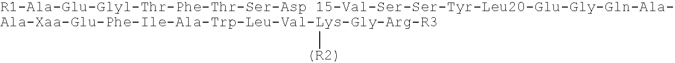

wherein R1 is selected from the group consisting of 4-imidazopropionyl, 4-imidazoacetyl, or 4-imidazo-a, a dimethyl-acetyl; R2 is selected from the group consisting of C6 Clo unbranched acyl, or is absent; P3 is selected from the group consisting of Gly-OH or NH2; and, Xaa is Lys or Arg, may be used in present invention.

Further preferred GLP-1 molecules capable of activating the GLP-1 receptor and suitable for use in the present invention include, but are not restricted to, any of the following molecules, which have previously been demonstrated to be capable of activating the GLP-1 receptor:
GLP-1 (7-36)amide
GLP-1 (7-37)
exendin-4
GLP-1 (7-34)
GLP-1 (7-35)
GLP-1 (7-36)
Gln9-GLP-1 (7-37)
D-Gln9-GLP-1 (7-37)
Thr16-Lys18-GLP-1 (7-37)
Lys18-GLP-1 (7-37)
GLP-1 (7-34)
GLP-1 (7-35)
GLP-1 (7-36)
GLP-1 (7-36)NH2
Gly8-GLP-1 (7-36)NH2
Gln9-GLP-1 (7-37)
D-Gln9-GLP-1 (7-37)
acetyl-Lys9-GLP-1 (7-37)
Thr9-GLP-1 (7-37)
D-Thr9-GLP-1 (7-37)
Asn9-GLP-1 (7-37)
D-Asn9-GLP-1 (7-37)
Ser22-Arg23-Arg24-Gln26-GLP-1 (7-37)
Thr16-Lys18-GLP-1 (7-37)
Lys18-GLP-1 (7-37)
Arg23-GLP-1 (7-37)
Arg24-GLP-1 (7-37)
GLP-1 (7-34)
GLP-1 (7-35)
GLP-1 (7-36) NH2
Gln9-GLP-1 (7-37)
d-Gln9-GLP1(7-37)
Thr16-Lys18-GLP-1 (7-37)
Lys18-GLP-1 (7-37)
Gly′-GLP-1 (7-36) NH2
Gly′-GLP1 (7-37) OH, Val′-GLP-1 (7-37) OH, Met8-GLP-1 (7-37) OH, acetyl-Lys9-GLP-1 (7-37)
Thr9 GLP-1 (7-37)
D-Thr9-GLP-1 (7-37)
Asn9-GLP-1 (7-37)
D-Asn9-GLP-1 (7-37)
Ser22-Arg23 Arg24-Gln26-GLP-1 (7-37)
Arg23-GLP-1 (7-37)
Arg24-GLP-1 (7-37)
a-methyl-Ala8-GLP-1 (736) NH2
Gly′-Gln2′-GLP-1 (7-37) OH
LY315902, (the activity of which is described in Naslund et al., “Glucagon-like peptide-1 analogue LY315902: effect on intestinal motility and release of insulin and somatostatin”, Regul Pept. 2002 Jun. 15; 106(1-3):89-95)

In another preferred embodiment of the present invention, said GLP-1 molecule is selected from any of the following molecules, which have previously been demonstrated to be capable of activating the GLP-1 receptor and are described by Xiao et al., “Biological Activities of Glucagon-Like Peptide-1 Analogues in Vitro and in Vivo, Biochemistry, 2001, Vol. 40, No. 9, p 2860-2869:
Acetyl-GLP-1
Hexanoic-GLP-1
(dAla2)GLP-1
(Gly2)GLP-1
(Ala4)GLP-1
Hexanoic-(dAla2)GLP-1
Glucagon(1-5)GLP-1
Glucagon(1-10)GLP-1
PACAP(1-5)GLP-1
PACAP(1-10)GLP-1
PHI(1-5)GLP-1
PHI(1-10)GLP-1
Secretin(1-10)GLP-1
VIP(1-5)GLP-1
VIP(1-10)GLP-1
(Ala12/16)GLP-1
(Ala8/11/16)GLP-1
(Ala8/12/16)GLP-1
(dHis1)GLP-1
desaminoHis1 GLP-1
N-me-His1-GLP-1
Alpha-me-His1-GLP-1
Imi-His1-Glp-1
(Ser2)GLP1

Further suitable GLP-1 molecules are those described in any of the following patent applications, hereby incorporated by reference thereto:
WO91/11457
U.S. Pat. No. 5,118,666
WO 20041094461 (“Human glucagons-like peptide-1 mimics and their use in the treatment of diabetes and related conditions”)
WO 98/08873, “Use of Glucagon-like peptide-1 (GLP-1) or analogs to abolish catabolic changes after surgery”
U.S. Pat. No. 5,545,618
U.S. Pat. No. 5,188,666
U.S. Pat. No. 5,512,549
WO 03/018516 (“Glucagon-like peptide-1 analogs”)
U.S. Pat. No. 5,120,712

### Homologues of GLP-1 Molecules

A homologue of one or more of the sequences specified herein may vary in one or more amino acids as compared to the sequences defined, but is capable of performing the same function, i.e. a homologue may be envisaged as a functional equivalent of a predetermined sequence.

Thus, in one preferred embodiment of the present invention, the GLP-1 molecule is a homologue of any of the molecules disclosed herein, such a homologue of any of the molecules selected from the group consisting of:
GLP-1(7-36)amide
GLP-1(7-37)
Liraglutide
Exenatide (Byetta)
Albugon
CJC-1131
zp-10-AVE0010
BIM51077 (Ipsen)
LY315902
LY307161
S 23521

Thus, in one preferred embodiment of the present invention, the GLP-1 molecule is a peptide containing one or more amino acid substitutions, inversion, additions or deletions, compared with a molecule selected from the group consisting of:
GLP-1(7-36)amide
GLP-1(7-37)
Liraglutide
Exenatide (Byetta)
Albugon
CJC-1131
zp-10-AVE0010
BIM51077 (Ipsen)
LY315902
LY307161
S 23521

In one embodiment, the number of substitutions, deletions, or additions is 20 amino acids or less, such as 15 amino acids or less, for example 10 amino acids or less, such as 9 amino acids or less, for example 8 amino acids or less such as 7 amino acids or less, for example 6 amino acids or less, such as 5 amino acids or less, for example 4 amino acids or less, such as 3 amino acids or less, for example 2 amino acids or less (such as 1), or any integer in between these amounts. In one aspect of the invention, the substitutions include one or more conservative substitutions, such as 20 or fewer conservative substitutions, for example 18 or fewer, such as 16 or fewer, for example 14 or fewer, such as 12 or fewer, for example 10 or fewer, such as 8 or fewer, for example 6 or fewer, such as 4 or fewer, for example 3 or fewer, such as 2 or fewer conservative substitutions. A “conservative” substitution denotes the replacement of an amino acid residue by another, biologically active similar residue. Examples of conservative substitution include the substitution of one hydrophobic residue, such as isoleucine, valine, leucine or methionine for another, or the substitution of one polar residue for another, such as the substitution of arginine for lysine, glutamic for aspartic acids, or glutamine for asparagine, and the like. The following table lists illustrative, but non-limiting, conservative amino acid substitutions.

| ORIGINAL RESIDUE | EXEMPLARY SUBSTITUTIONS |
|---|---|
| ALA | SER, THR, VAL, GLY |
| ARG | LYS |
| ASN | HIS, SER |
| ASP | GLU, ASN |
| CYS | SER |
| GLN | ASN, HIS |
| GLU | ASP, GLU |
| GLY | ALA, SER |
| HIS | ASN, GLN |
| ILE | LEU, VAL, THR |
| LEU | ILE, VAL |
| LYS | ARG, GLN, GLU, THR |
| MET | LEU, ILE, VAL |
| PHE | LEU, TYR |
| SER | THR, ALA, ASN |
| THR | SER, ALA |
| TRP | ARG, SER |
| TYR | PHE |
| VAL | ILE, LEU, ALA |
| PRO | ALA |

Other GLP-1 homologues suitable for the uses and methods of the present invention are peptide sequences having greater than 50 percent sequence identity, and preferably greater than 90 percent sequence identity (such as greater than 91% sequence identity, for example greater than 92% sequence identity, such as greater than 93% sequence identity, for example greater than 94% sequence identity, such as greater than 95% sequence identity, for example greater than 96% sequence identity, such as greater than 97% sequence identity, for example greater than 98% sequence identity, such as greater than 99% sequence identity, for example greater than 99.5% sequence identity), to a molecule selected from the group consisting of:
GLP-1(7-36)amide
GLP-1(7-37)
Liraglutide
Exenatide (Byetta)
Albugon
CJC-1131
zp-10-AVE0010
BIM51077 (Ipsen)
LY315902
LY307161
S 23521

As used herein, sequence identity refers to a comparison made between two molecules using standard algorithms well known in the art. The preferred algorithm for calculating sequence identity for the present invention is the Smith-Waterman algorithm, where the reference sequence is used to define the percentage identity of polypeptide homologs over its length. The choice of parameter values for matches, mismatches, and inserts or deletions is arbitrary, although some parameter values have been found to yield more biologically realistic results than others. One preferred set of parameter values for the Smith-Waterman algorithm is set forth in the “maximum similarity segments” approach, which uses values of 1 for a matched residue and −⅓ for a mismatched residue (a residue being either a single nucleotide or single amino acid) (Waterman, Bull. Math. Biol. 46, 473-500 (1984)). Insertions and deletions (indels), x, are weighted as

xₖ=1+k/3,

where k is the number of residues in a given insert or deletion (Id.).

For instance, a sequence that is identical to a 42 amino acid residue sequence, except for 18 amino acid substitutions and an insertion of 3 amino acids, would have a percent identity given by:

[(1×42 matches)−(1/3×18 mismatches)−(1+3/3 indels)]/42=81% identity

In one preferred embodiment of the present invention, truncations at the end of the molecule are not taken into account when calculating sequence identity (i.e., if one molecule is longer than the other, only the overlapping lengths of the molecules are used in the sequence identity analysis); in another preferred embodiment of the present invention, truncations are counted as deletions.

A GLP-1 homologue may include the D-amino acid forms and may be a molecule having one or more amino acid substitutions, deletions, inversions, or additions relative to a molecule selected from the group consisting of:
GLP-1(7-36)amide
GLP-1(7-37)
Liraglutide
Exenatide (Byetta)
Albugon
CJC-1131
zp-10-AVE0010
BIM51077 (Ipsen)
LY315902
LY307161
S 23521

In one preferred embodiment of the present invention, the GLP-1 molecule is a peptide containing one or more amino acid substitutions, inversion, additions or deletions, compared with GLP-1 (7-36) amide. In one embodiment, the number of substitutions, deletions, or additions is 20 amino acids or less, such as 15 amino acids or less, for example 10 amino acids or less, such as 9 amino acids or less, for example 8 amino acids or less such as 7 amino acids or less, for example 6 amino acids or less, such as 5 amino acids or less, for example 4 amino acids or less, such as 3 amino acids or less, for example 2 amino acids or less (such as 1), or any integer in between these amounts. In one aspect of the invention, the substitutions include one or more conservative substitutions. Examples of suitable conservative substitutions are given above.

Other GLP-1 homologues suitable for the uses and methods of the present invention are peptide sequences having greater than 50 percent sequence identity, and preferably greater than 90 percent sequence identity (such as greater than 91% sequence identity, for example greater than 92% sequence identity, such as greater than 93% sequence identity, for example greater than 94% sequence identity, such as greater than 95% sequence identity, for example greater than 96% sequence identity, such as greater than 97% sequence identity, for example greater than 98% sequence identity, such as greater than 99% sequence identity, for example greater than 99.5% sequence identity), to (1) SEQ ID NOS:1, 2, 3, 4; and/or (2) to truncated sequences thereof. As used herein, sequence identity refers to a comparison made between two molecules using standard algorithms well known in the art. The preferred algorithm for calculating sequence identity for the present invention is the Smith-Waterman algorithm, as described above.

A GLP-1 homologue may also be a molecule having one or more amino acid substitutions, deletions, inversions, or additions relative to human GLP-1 (7-37) and may include the D-amino acid forms.

In another embodiment of the present invention, said homologue of any of the predetermined sequences herein, such as any of SEQ ID NO: 1-4, may be defined as:
i) homologues comprising an amino acid sequence capable of binding selectively to the human GLP-1 receptor, and/or
ii) homologues having a substantially similar or higher binding affinity to the GLP-1 receptor than human GLP-1.

In another preferred embodiment, the GLP-1 molecule is an antibody raised against the GLP-1 receptor.

### Chemically Derivatized GLP-1 Molecules

It is further understood that GLP-1 molecules suitable for use in the present invention may be chemically derivatized or altered, for example, peptides with non-natural amino acid residues (e.g., taurine residue, beta- and gamma-amino acid residues and D-amino acid residues), C-terminal functional group modifications, such as amides, esters, and C-terminal ketone modifications and N-terminal functional group modifications, such as acylated amines, Schiff bases, or cyclization, such as found, for example, in the amino acid pyroglutamic acid. One example of a derivatized molecules is Liraglutide.

### Protected GLP-1 Molecules

Furthermore, because the enzyme, dipeptidyl-peptidase IV (DPP IV), may be responsible for the observed rapid in vivo inactivation of administered GLP-1, [see, e.g., Mentlein, R., et al., Eur. J. Biochem., 214:829-835 (1993)], administration of GLP-1 molecules that are protected from the activity of DPP IV may in some embodiments be preferred. Thus, in one preferred embodiment of the present invention, a DPP-IV protected GLP-1 molecule can be used. “DPP-IV protected GLPs” refers to GLP-1 analogs which are resistant to the action of DPP-IV. These include analogs having a modified or D amino acid residue in position 8 and includes biosynthetic GLP-1 analogs having Gly, Val, Thr, Met, Ser, Cys, or Asp in position 8. Other DPP-IV protected GLPs include desamino His derivatives. Preferred embodiments include any of the following: Gly8-GLP-1 (7-36)NH2, Val8-GLP-1(7-37)OH, a-methyl-Ala8-GL1(7-36)NH2, and Gly8-Gln21-GLP-1(7-37)OH, or pharmaceutically acceptable salts thereof.

### GLP-1 Molecule Conjugates

The GLP-1 molecules of the present invention may also be modified with reactive groups capable of forming covalent bonds to yield GLP-1 compounds that are capable of being conjugated to blood components so as to stabilize the GLP-1 molecule. Suitable examples are described in WO 03/103572 (“Modified Glucagon-like peptide-1 analogs”) and US 2004/0138100 (Long lasting synthetic glucagons like peptide (GLP-1).

Thus, in one embodiment of the present invention, the GLP-1 molecule used is a GLP-1 peptide modified with an activated disulfide bond group or S-sulfonate

Other suitable GLP-1 molecules for use in the present invention include those disclosed in WO 00/34331 (“Analogues of GLP-1”), and WO 01/98331 (“Glucagon-like peptide-1 analogs”), the contents of which are incorporated herein by reference.

Further suitable GLP-molecules for use in the present invention are disclosed in WO 00/37098 (“Shelf-stable formulation of Glucagon-like peptide-1”), the contents of which are incorporated by reference herein.

### Particularly Preferred GLP-1 Molecules for Use in the Present Invention:

Particularly preferred GLP-1 molecules for use in the present invention are selected from any of the following:

### Liraglutide

LIRAGLUTIDE (with chemical structure Arg(34)Lys(26)-(N-epsilon-(gamma-Glu(N-alpha-hexadecanoyl))-GLP-1(7-37)). Preferably, Liraglutide is administered subcutaneously. Preferred doses of Liraglutide are 0.045 mg, 0.225 mg, 0.45 mg, 0.60 mg, or 0.75 mg, such as 0.6 mg. In another preferred embodiment, a dosage of up to 200 ug/kg may be administered, such as up to twice daily, for example 1.25-20 ug/kg*d.

### Exanatide

Exanatide (Amylin Corporation, (see J Biol Chem 1990; 265:20259-62). Exanatide displays similar properties to native GLP-1, and regulates gastric emptying, insulin secretion, food intake, and glucagon secretion.

Exanatide is much more potent than native GLP-1, largely due to its resistance to DPP-IV-mediated inactivation. In contrast to GLP-1 which contains an alanine at position 2, exendin-4 has a position 2 glycine, hence it is not a substrate for DP IV and has a much longer t1/2 (half-life) in vivo. In the methods of the present invention, it is preferred that Exanatide is administered in a dosage of 5 micrograms or 10 micrograms, preferably on a daily basis.

### Albuaon and CJC-1131

Albugon (GlaxoSmithKline/Human Genome Sciences) and CJG-1131 (Conjuchem Inc.) are GLP-1-albumin proteins which exploit the long circulating half life of albumin to extend the short duration of action of native GLP-1. Whereas CJC-1131 is a human GLP-1 analogue that forms a covalent bond with human serum albumin following subcutaneous injection of the free CJC-1131 peptide in vivo, Albugon is a recombinant GLP-1-albumin protein produced ex vivo prior to administration of the much larger single recombinant protein in vivo.

Albugon has been shown to inhibit gastric emptying and inhibit food intake in mice following both icv and peripheral administration. (Diabetes. 2004 September; 53(9):2492-500). In one preferred embodiment of the present invention, Albugon is administered in a (preferably daily) dosage of 150-350 ug/kg. CJC-1131 is a human GLP-1 analogue, modified to be resistant to DPP-IV, with a reactive chemical linker at the carboxy terminal end of the molecule which permits covalent coupling to albumin (Cys 34 residue) following administration in vivo. As albumin exhibits a long circulating half life in vivo, albumin-conjugated drugs should exhibit prolonged action, and delayed clearance, consistent with the known turnover of albumin in human subjects, which is estimated to exhibit a t1/2 of about 15-19 days.

CJC-1131 has the ability to bind and activate the GLP-1 receptor both in vitro and in vivo, and studies in normal and diabetic rodents demonstrate that CJC-1131 exhibits GLP-1-dependent activities (Diabetes. 2003 March; 52(3):751-9). In one embodiment of the present invention, it is preferred that CJC-1131 is administered in a dosage of 2.1-2.6 ug/kg per day. In another preferred embodiment of the present invention, it is preferred that CJC-1131 is administered in a (preferably daily) dosage of 150-350 ug/kg.

Other particularly preferred GLP-1 molecules for use in the present invention include, but are not restricted to, any of the following: zp-10 (see e.g. J Pharmacol Exp Ther. 2003 November; 307(2):490-6), BIM51077 (Ipsen), LY315902 (Regul Pept. 2002 Jun. 15; 106(1-3):89-95.), LY307161 (Eli Lilly), S 23521 (J Endocrinol. 2005 March; 184(3):505-13).

### Functional Activity of GLP-1 Molecule

The GLP-1 molecules useful in the inventive methods and uses described herein are active at the GLP-1 receptor. The GLP-1 molecules can bind to the receptor, and preferably, stimulate receptor activity.

The receptor activity can be measured using different techniques such as detecting a change in the intracellular conformation of the receptor, in the G-protein coupled activities, and/or in the intracellular messengers.

One simple measure of the ability of a GLP-1 molecule to activate the GLP-1 receptor is to measure its EC50, i.e. the dose at which the compound is able to activate the signalling of the receptor to half of the maximal effect of the compound. The receptor can either be expressed endogenously on primary cells cultures, for example pituitary cells, or heterologously expressed on cells transfected with the ghrelin receptor. Whole cell assays or assays using membranes prepared form either of these cell types can be used depending on the type of assay.

GLP-1 biological activity can be determined by standard methods, in general, by receptor-binding activity screening procedures which involve providing appropriate cells that express the GLP-1 receptor on their surface, for example, insulinoma cell lines such as RINmSF cells or INS-1 cells. See also Mojsov, Int J Pept Protein Res 40, 333-43 (1992) and EP0708170A2. Cells that are engineered to express a GLP-1 receptor also can be used. In addition to measuring specific binding of tracer to membrane using radioimmunoassay methods, cAMP activity or glucose dependent insulin production can also be measured. In one method, a polynucleotide encoding the GLP-1 receptor is employed to transfect cells to thereby express the GLP-1 receptor protein. Thus, for example, these methods may be employed for screening for a receptor agonist by contacting such cells with compounds to be screened and determining whether such compounds generate a signal, i.e activate the receptor.

Polyclonal and monoclonal antibodies can be utilized to detect purify and identify GLP-1 like molecules for use in the methods described herein. Antibodies such as ABGA1178 detect intact unprocessed GLP-1(137) or N-terminally-truncated GLP-1(7-37) or (7-36)amide. Other antibodies detect on the very end of the C-terminus of the precursor molecule, a procedure which allows by subtraction to calculate the amount of biologically active truncated peptide, i.e. GLP-1 (7-37)amide (Orskov et al., Diabetes, 42, 658-661 (1993); Orskov et al., J. Clin. Invest. 87, 415-423 (1991)).

Other screening techniques include the use of cells which express the GLP-1 receptor, for example, transfected CHO cells, in a system which measures extracellular pH or ionic changes caused by receptor activation. For example, potential agonists may be contacted with a cell which expresses the GLP-1 protein receptor and a second messenger response, e.g. signal transduction or ionic or pH changes, may be measured to determine whether the potential agonist is effective.

In one embodiment the binding of a GLP-1 molecule to the GLP-1 receptor can be measured by the use of the assay described herein above.

A GLP-1 molecule for use according to the invention preferably has at least about 50%, at least about 60%, at least about 70%, at least about 80%, or at least about 90%, functional activity relative to human GLP-1 as determined using the assay described herein above, and/or an EC50 greater than about 1,000, greater than about 100, or greater than about 50, or greater than about 10. Greater refers to potency and thus indicates a lesser amount is needed to achieve binding inhibition.

In one embodiment of the invention, the compound has a potency (EC50) on the GLP-1 receptor of less than 500 nM. In another embodiment the compound has a potency (EC50) on the GLP-1 receptor of less than 100 nM, such as less than 80 nM, for example less than 60 nM, such as less than 40 nM, for example less than 20 nM, such as less than 10 nM, for example less than 5 nM, such as less than 1 nM, for example less than 0.5 nM, such as less than 0.1 nM, for example less than 0.05 nM, such as less than 0.01 nM.

In a further embodiment the dissociation constant (Kd) of the compound is less than 500 nM. In a still further embodiment the dissociation constant (Kd) of the ligand is less than 100 nM, such as less than 80 nM, for example less than 60 nM, such as less than 40 nM, for example less than 20 nM, such as less than 10 nM, for example less than 5 nM, such as less than 1 nM, for example less than 0.5 nM, such as less than 0.1 nM, for example less than 0.05 nM, such as less than 0.01 nM.

Binding assays can be performed using recombinantly-produced receptor polypeptides present in different environments. Such environments include, for example, cell extracts and purified cell extracts containing the receptor polypeptide expressed from recombinant nucleic acid or naturally occurring nucleic acid; and also include, for example, the use of a purified GLP-1 receptor polypeptide produced by recombinant means or from naturally occurring nucleic acid which is introduced into a different environment.

Using a recombinantly expressed GLP-1 receptor offers several advantages such as the ability to express the receptor in a defined cell system, so that a response to a compound at the receptor can more readily be differentiated from responses at other receptors. For example, the receptor can be expressed in a cell line such as HEK 293, COS 7, and CHO not normally expressing the receptor by an expression vector, wherein the same cell line without the expression vector can act as a control.

### Pharmaceutically Acceptable GLP-1 Salts

A pharmaceutically-acceptable salt form of any of the GLP-1 molecules described herein may be used in the uses and methods of the present invention. Acids commonly employed to form acid addition salts are inorganic acids such as hydrochloric acid, hydrobromic acid,hydroiodic acid, sulfuric acid, phosphoric acid, and the like, and organic acids such as ptoluenesulfonic acid, methanesulfonic acid, oxalic acid, pbromophenyl-sulfonic acid, carbonic acid, succinic acid, citric acid, benzoic acid, acetic acid, and the like.

Examples of such salts include thesulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like. Preferred acid addition salts are those formed with mineral acids such as hydrochloric acid and hydrobromic acid, and, especially, hydrochloric acid.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like. The salt forms are particularly preferred.

A GLP-1 molecule suitable to be used in the present invention may be formulated with one or more excipients before use in the present invention. For example, the active compound used in the present invention may be complexed with a divalent metal cation by well-known methods. Such metal cations include, for example, Zn++, Mn++, Fe++, Co++, Cd++, Ni++, and the like.

Optionally, the active compound used in the present invention may be combined with a pharmaceutically-acceptable buffer, and the pH adjusted to provide acceptable stability, and a pH acceptable for parenteral administration.

Optionally, one or more pharmaceutically-acceptable anti-microbial agents may be added. Meta-cresol and phenol are preferred pharmaceutically-acceptable anti-microbial agents. One or more pharmaceutically-acceptable salts may be added to adjust the ionic strength or tonicity. One or more excipients may be added to further adjust the isotonicity of the formulation. Glycerin is an example of an isotonity adjusting excipient.

### Manufacture of the GLP-1 Molecules for Use in the Present Invention

The GLP-1 molecules suitable for use in the present invention may be manufactured using any suitable method known to one skilled in the art.

For example, the GLP-1 molecules of the invention that are peptides can be made by solid state chemical peptide synthesis. Such peptides can also be made by conventional recombinant techniques using standard procedures described in, for example, Sambrook & Maniatis. “Recombinant”, as used herein, means that a gene is derived from a recombinant (e.g., microbial or mammalian) expression system which has been genetically modified to contain polynucleotide encoding a GLP-1 molecule as described herein. The GLP-1 like peptides can be recovered and purified from recombinant cell cultures by methods including, but not limited to, ammonium sulfate or ethanol precipitation, acid extraction, anion or cation exchange chromatography, phosphocellulose chromatography, hydrophobic interaction chromatography, affinity chromatography) hydroxylapatite chromatography and lectin chromatography. High performance liquid chromatography (HPLC) can be employed for final purification steps. The GLP-1 molecule peptides of the present invention may be a naturally purified product, or a product of chemical synthetic procedures, or produced by recombinant techniques from prokaryotic or eukaryotic hosts (for example by bacteria, yeast, higher plant, insect and mammalian cells in culture or in vivo). Depending on the host employed in a recombinant production procedure, the polypeptides of the present invention are generally non-glycosylated, but may be glycosylated.

Other suitable methods for preparation of a GLP-1 molecule include the recombinant method described in WO 03/046158 (“A Method for preparing the Recombinant Human Glucagon like peptide-1 (7-37)”), the contents of which are incorporated herein by reference.

The GLP-1 molecule may also be solubilized using e.g. any of the methods described in WO 01/55213 (“Process for solubilizing glucagon-like peptide 1 compounds”), the contents of which are incorporated herein by reference.

### Pharmaceutical Formulation

While it is possible for the compounds or salts of the present invention to be administered as the raw chemical, it is preferred to present them in the form of a pharmaceutical composition, for use in any of the uses or methods described herein.

Thus, in one embodiment of the present invention, the GLP-1 molecule is formulated in a shelf-stable pharmaceutical formulation comprising a therapeutically effective amount of the GLP-1 molecule(s), a pharmaceutically acceptable preservative, and a tonicity modifier. In another preferred embodiment, the formulation comprises a surfactant, such as Brij-35.

In one embodiment the invention relates to a pharmaceutical composition comprising a mixture of at least two different GLP-1 molecules, such as a mixture of a short acting GLP-1 molecule type with a longer-acting GLP-1 molecule, in order to obtain both an immediate and sustained effect. Without being bound by theory it is believed that such a mixture will have a longer half-life in plasma. The pharmaceutical composition may comprise any GLP-1 molecule or a pharmaceutically acceptable salt thereof and pharmaceutically acceptable carriers, vehicles and/or excipients, said composition further optionally comprising transport molecules.

### Transport Molecules

Transport molecules may be added in order to increase the half-life of the GLP-1 molecule. Transport molecules act by having incorporated into or anchored to it the compound according to the invention. Any suitable transport molecules known to the skilled person may be used. Preferred examples are liposomes, micelles, and/or microspheres.

Conventional liposomes are typically composed of phospholipids (neutral or negatively charged) and/or cholesterol. The liposomes are vesicular structures based on lipid bilayers surrounding aqueous compartments. They can vary in their physiochemical properties such as size, lipid composition, surface charge and number and fluidity of the phospholipids bilayers. The most frequently used lipid for liposome formation are: 1,2-Dilauroyl-sn-Glycero-3-Phosphocholine (DLPC), 1,2-Dimyristoyl-sn-Glycero-3-Phosphocholine (DMPC), 1,2-Dipalmitoyl-sn-Glycero-3-Phosphocholine (DPPC), 1,2-Distearoyl-sn-Glycero-3-Phosphocholine (DSPC), 1,2-Dioleoyl-sn-Glycero-3-Phosphocholine (DOPC), 1,2-Dimyristoyl-sn-Glycero-3-Phosphoethanolamine (DMPE),1,2-Dipalmitoyl-sn-Glycero-3-Phosphoethanolamine (DPPE),1,2-Dioleoyl-sn-Glycero-3-Phosphoethanolamine (DOPE),1,2-Dimyristoyl-sn-Glycero-3-Phosphate (Monosodium Salt) (DMPA), 1,2-Dipalmitoyl-sn-Glycero-3-Phosphate (Monosodium Salt) (DPPA), 1,2-Dioleoyl-sn-Glycero-3-Phosphate (Monosodium Salt) (DOPA), 1,2-Dimyristoyl-sn-Glycero-3-[Phospho-rac-(1-glycerol)] (Sodium Salt) (DMPG), 1,2-Dipalmitoyl-sn-Glycero-3-[Phospho-rac-(1-glycerol)] (Sodium Salt) (DPPG), 1,2-Dioleoyl-sn-Glycero-3-[Phospho-rac-(1-glycerol)] (Sodium Salt) (DOPG), 1,2-Dimyristoyl-sn-Glycero-3-[Phospho-L-Serine] (Sodium Salt) (DMPS), 1,2-Dipaimitoyl-sn-Glycero-3-[Phospho-L-Serine) (Sodium Salt) (DPPS), 1,2-Dioleoyl-sn-Glycero-3-[Phospho-L-Serine] (Sodium Salt) (DOPS), 1,2-Dioleoyl-sn-Glycero-3-Phosphoethanolamine-N-(glutaryl) (Sodium Salt) and 1,1′,2,2′-Tetramyristoyl Cardiolipin (Ammonium Salt). Formulations composed of DPPC in combination with other lipid or modifiers of liposomes are preferred e.g. in combination with cholesterol and/or phosphatidyloholine.

Long-circulating liposomes are characterized by their ability to extravasate at body sites where the permeability of the vascular wall is increased. The most popular way to produce long circulating liposomes is to attach hydrophilic polymer polyethylene glycol (PEG) covalently to the outer surface of the liposome. Some of the preferred lipids are: 1,2-Dipalmitoyl-sn-Glycero-3-Phosphoethanolamine-N-[Methoxy(Polyethylene glycol)-2000] (Ammonium Salt), 1,2-Dipalmitoyl-sn-Glycero-3-Phosphoethanolamine-N-[Methoxy(Polyethylene glycol)-5000] (Ammonium Salt), 1,2-Dioleoyl-3-Trimethylammonium-Propane (Chloride Salt) (DOTAP).

Possible lipid applicable for liposomes are supplied by Avanti, Polar lipids, Inc, Alabaster, Ala. Additionally, the liposome suspension may include lipid-protective agents which protect lipids against free-radical and lipid-peroxidative damages on storage. Lipophilic free-radical quenchers, such as alpha-tocopherol and water-soluble iron-specific chelators, such as ferrioxianine, are preferred.

A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028, all of which are incorporated herein by reference. One methode is described in example 9. Another method produces multilamellar vesicles of heterogeneous sizes. In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powderlike form. This film is covered with an aqueous solution of the targeted drug and the targeting component and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents such as deoxycholate.

Micelles are formed by surfactants (molecules that contain a hydrophobic portion and one or more ionic or otherwise strongly hydrophilic groups) in aqueous solution.

Common surfactants well known to one of skill in the art can be used in the micelles of the present invention. Suitable surfactants include sodium laureate, sodium oleate, sodium lauryl sulfate, octaoxyethylene glycol monododecyl ether, octoxynol 9 and PLURONIC F-127 (Wyandotte Chemicals Corp.). Preferred surfactants are nonionic polyoxyethylene and polyoxypropylene detergents compatible with IV injection such as, TWEEN-80., PLURONIC F-68., n-octyl-.beta.-D-glucopyranoside, and the like. In addition, phospholipids, such as those described for use in the production of liposomes, may also be used for micelle formation.

### Buffer and Other Excipients

GLP-1 molecules themselves exhibit a buffering capacity. However, to maintain the pH of the composition for long term storage and stability, it is preferable to add a buffer, such as TRIS. In one preferred embodiment, the formulation has a pH that is about 8.2 to about 8.8, such as about 8.3 to about 8.6, for example about 8.4 to about 8.5. As used in this specification with respect to pH, the term “about” means plus or minus 0.1 pH units. Thus, a pH of “about 8.5” denotes a pH of 8.4 to 8.6. The buffers which are used may be e.g. tromethane (TRIS), and amino-acid based buffers such as lysine and hydroxy-lysine. The term “TRIS” refers to 2-amino-2-hydroxymethyl-1,3-propanediol (also known in the art as tromethane, trimethylol aminomethane or tris (hydroxymethyl) aminomethane), and to any pharmacologically acceptable salt thereof. The free base and the hydrochloride form are two common forms of TRIS.

The concentration of the GLP-1 molecule that is used in the inventive formulation is in one preferred embodiment about 0.30 to about 0.65 mg/ml of the GLP-1 molecule, such as about 0.5 mg/ml of a GLP-1 molecule.

The GLP-1 molecule may also be formulated with a preservative, such as a phenolic preservative, for example m-cresol, phenol, benzyl alcohol, or methyl paraben. One preferred amount of preservative is from about 2 mg/ml to about 6 mg/ml. However, one skilled in the art is aware that the concentration of preservative necessary for effective preservation depends on the preservative used, the pH of the formulation, and whether substances that bind or sequester the preservative are also present. Preferably, m-cresol is used in the formulations as a preservative.

While a buffer and a preservative are most preferably included in the formulation, other additional excipients may be included, such as a tonicity modifier and/or a surfactant as well as distilled water for injections. The tonicity modifier may be included to make the formulation approximately isotonic with bodily fluid depending on the mode of administration. The concentration of the tonicity modifier is in accordance with the known concentration of a tonicity modifier in a peptide formulation. A preferable tonicity modifier used in the present invention is glycerol.

### Dosing Regimes

Suitable dosing regimens for the various compounds and methods of the present invention are preferably determined taking into account factors well known in the art including type of subject being dosed; age, weight, sex and medical condition of the subject; the route of administration; the renal and hepatic function of the subject; the desired effect; and the particular compound employed.

Optimal precision in achieving concentrations of drug within the range that yields efficacy without toxicity requires a regimen based on the kinetics of the drug's availability to target sites. This involves a consideration of the distribution, equilibrium, and elimination of a drug.

A typical dosage of a compound employed according to the invention is in a concentration equivalent to from 10 ng to 10 mg GLP-1 molecule per kg bodyweight. The concentrations and amounts herein are given in equivalents of amount human GLP-1, Equivalents may be tested as described in the section entitled “Functionality”, above.

In a preferred embodiment the medicament is administered in a concentration equivalent to from 0.1 μg to 1 mg human GLP-1 per kg bodyweight, such as from 0.5 μg to 0.5 mg human GLP-1 per kg bodyweight, such as from 1.0 μg to 0.1 mg human GLP-1 per kg bodyweight, such as from 1.0 μg to 50 μg human GLP-1 per kg bodyweight, such as from 1.0 μg to 10 μg human GLP-1 per kg bodyweight.

In one embodiment of the present invention, the dosage unit for use in treating biliary dyskinesia and/or biliary pain in a patient comprises any of the GLP-1 molecules described herein and a pharmaceutically suitable excipient. Preferably, the dosage unit is in the range of about 0.4 to 2.4 pmol kg⁻¹ min⁻¹. Still more preferably, the dosage unit is in the range of about 0.8 to 1.2 pmol kg⁻¹ min⁻¹. For the purpose of this invention, the term “about” is defined as +/−10%. For example, a dosage unit in the range of about 0.4 to 2.4 pmol kg⁻¹ min⁻¹ means 0.196 to 2.64 pmol kg⁻¹ min⁻¹.

The composition of the present invention can be used as a systematic or local application by oral or parenteral administration. Alternatively, the composition may be applied as an intravenous or subcutaneous injection. Preferably, the dosage unit is in the range of about 0.4 to 2.4 pmol kg⁻¹ min⁻¹. Still more preferably, the dosage unit is in the range of about 0.8 to 1.2 pmol kg⁻¹ min⁻¹.

### Solid Formulations

The GLP-1 molecule may be prepared as a solid composition for oral administration—including tablets, preparations, granules and the like. In such a solid composition, one or more active ingredients may be mixed with at least one inactive diluent, for example, lactose, mannitol, glucose, hydroxypropyl cellulose, microcrystalline cellulose, starch, polyvinyl pyrrolidone, magnesium aluminate metasilicate and the like. According to the usual work-up, the composition may contain additives other than inactive diluent, for example, lubricant such as magnesium stearate; disintegrant such as fibrous calcium gluconate; stabilizer such as cyclodextrin, for example, alpha, beta- or gamma-cyclodextrin; etherified cyclodextrin such as dimethyl-alpha-, dimethyl-beta-, trimethyl-beta-, or hydroxypropyl-beta-cyclodextrin; branched cyclodextrin such as glucosyl-, maltosyl-cyclodextrin; formylated cyclodextrin, cyclodextrin containing sulfur; phospholipid and the like. When the above cyclodextrins are used, inclusion compound with cyclodextrins may be sometimes formed to enhance stability. Alternatively, phospholipid may be sometimes used to form liposome, resulting in enhanced stability.

Tablets or pills may be coated with film soluble in the stomach or intestine such as sugar, gelatin, hyroxypropyl cellulose, hydroxypropylmethyl cellulose phthalate as needed. Further, they may be formed as capsules with absorbable substances such as gelatin.

### Liquid Compositions

A liquid composition for oral administration may contain pharmaceutically acceptable emulsion, solution, suspension, syrup, elixer as well as generally used inactive diluent. Such composition may contain, in addition to the inactive diluent, adjuvants such as lubricants and suspensions, sweetening agents, flavoring agents, preservatives, solubilizers, anti-oxidants and the like. The details of the additives may be selected from those described in any general textbook in the pharmaceutical field. Such liquid compositions may be directly enclosed in soft capsules.

Solutions for parenteral administration, for example, suppository, enema and the like according to the present invention include sterile, aqueous or non-aqueous solution, suspension, emulsion, detergent and the like. The aqueous solution and suspension includes, for example, distilled water, physiological saline and Ringer's solution.

The non-aqueous solution and suspension include, for example, propylene glycol, polyethylene glycol, fatty acid triglyceride, vegetable oil such as olive oil, alcohols such as ethanol, polysorbate and the like. Such composition may contain adjuvants such as preservatives, wetting agent, emulsifier, dispersant, anti-oxidants and the like.

### Administration Methods

Administration may be via any route known to be effective by the physician of ordinary skill. Parenteral administration is preferred. Parenteral administration is commonly understood in the medical literature as the injection of a dosage form into the body by a sterile syringe or some other mechanical device such as an infusion pump. Parenteral routes include intravenous, intramuscular, subcutaneous, intraperitoneal, intraspinal, intrathecal, nasal, pulmonary, buccal, intraarterial, intracerebroventricular, intraarterial, subarachnoid, and epidural. Intravenous, intramuscular, and subcutaneous routes of administration of the compounds used in the present invention are more preferred. Intravenous and subcutaneous routes of administration of the compounds used in the resent invention are yet more highly preferred. For parenteral administration, an active compound used in the present invention preferably is combined with distilled water at an appropriate pH.

Additional pharmaceutical methods may be employed to control the duration of action. Controlled release preparations may be achieved by the use of polymers to complex or absorb the active compound used in the present invention.

Extended duration may be obtained by selecting appropriate macromolecules, for example, polyesters, polyamino acids, polyvinylpyrrolidone, ethylenevinyl acetate, methylcellulose, carboxymethylcellulose, or protamine sulfate, and by selecting the concentration of macromolecules, as well as the methods of incorporation, in order to prolong release. Another possible method to extend the duration of action by controlled release preparations is to incorporate an active compound used in the present invention into particles of a polymeric material such as polyesters, polyamino acids, hydrogels, poly (lactic acid) or ethylene vinylacetate copolymers. Alternatively, instead of incorporating a compound into these polymeric particles, it is possible to entrap a compound used in the present invention in microcapsules prepared, for example, by coacervation techniques or by interfacial polymerization, for example, hydroxymethylcellulose or gelatin-microcapsules, respectively, or in colloidal drug delivery systems, for example, liposomes, albumin microspheres, microemulsions, nanoparticles, and nanocapsules, or in macroemulsions. Such teachings are disclosed in Remington's Pharmaceutical Sciences (1980).

One preferred route of administration is via a pen injection system, wherein for example either a 1.5 ml cartridge or a 3.0 ml cartridge is utilized.

### Nasal/Aerosol Administration

The compounds of the present invention may be formulated for nasal administration. The solutions or suspensions are applied directly to the nasal cavity by conventional means, for example with a dropper, pipette or spray. The compositions may be provided in a single or multidose form. In the latter case of a dropper or pipette this may be achieved by the patient administering an appropriate, predetermined volume of the solution or suspension. In the case of a spray this may be achieved for example by means of a metering atomizing spray pump.

The compounds of the present invention may be formulated for aerosol administration, particularly to the respiratory tract and including intranasal administration. The compound will generally have a small particle size for example of the order of 5 microns or less. Such a particle size may be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

Compositions administered by aerosols may be prepared, for example, as solutions in saline, employing benzyl alcohol or other suitable preservatives, absorption promoters to enhance bioavailability, employing fluorocarbons, and/or employing other solubilizing or dispersing agents.

### Compositions for Oral Administration

Those GLP-1 molecule types capable of remaining biologically active in an individual after oral administration (such as e.g. small molecules and short peptides) can be formulated in a wide range of oral administration dosage forms. The pharmaceutical compositions and dosage forms may comprise the compounds of the invention or its pharmaceutically acceptable salt or a crystal form thereof as the active component. The pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, wetting agents, tablet disintegrating agents, or an encapsulating material.

Preferably, the composition will be about 0.5% to 75% by weight of a compound or compounds of the invention, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like.

In powders, the carrier is a finely divided solid which is a mixture with the finely divided active component. In tablets, the active component is mixed with the carrier having the necessary binding capacity in suitable proportions and compacted in the shape and size desired. The powders and tablets preferably containing from one to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the composition of the active compound with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container aseptically. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing agents, and the like.

Other forms suitable for oral administration include liquid form preparations including emulsions, syrups, elixirs, aqueous solutions, aqueous suspensions, toothpaste, gel dentifrice, chewing gum, or solid form preparations which are intended to be converted shortly before use to liquid form preparations. Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavours, stabilizing and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavours, stabilizers, buffers, artificial and natural sweeteners, dispersants, thickeners, solubilizing agents, and the like.

### Compositions for Parenteral Administration

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water. Aqueous solutions should be suitably buffered if necessary, and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Solutions of ghrelin or a ghrelin-like compound or pharmaceutically acceptable salt thereof, (and for example antigenic epitopes and protease inhibitors) can be prepared in water or saline, and optionally mixed with a nontoxic surfactant. Compositions for intravenous or intra-arterial administration may include sterile aqueous solutions that may also contain buffers, liposomes, diluents and other suitable additives.

Oils useful in parenteral compositions include petroleum, animal, vegetable, or synthetic oils, Specific examples of oils useful in such compositions include peanut, soybean, sesame, cottonseed, corn, olive, petrolatum, and mineral. Suitable fatty acids for use in parenteral compositions include oleic acid, stearic acid, and isostearic acid. Ethyl oleate and isopropyl myristate are examples of suitable fatty acid esters.

Suitable soaps for use in parenteral compositions include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-.beta.-aminopropionates, and 2-alkyl-imidazoline quaternary ammonium salts, and (e) mixtures thereof.

The parenteral compositions typically will contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such compositions will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral compositions can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The pharmaceutical dosage forms suitable for injection or infusion can include sterile aqueous solutions or dispersions comprising the active ingredient that are adapted for administration by encapsulation in liposomes. In all cases, the ultimate dosage form must be sterile, fluid and stable under the conditions of manufacture and storage.

Sterile injectable solutions are prepared by incorporating ghrelin or a ghrelin-like compound or pharmaceutically acceptable salt thereof in the required amount in the appropriate solvent with various of the other ingredients enumerated above, as required, followed by filter sterilization.

### Compositions for Topical Administration

The compounds of the invention can also be delivered topically. Regions for topical administration include the skin surface and also mucous membrane tissues of the rectum, nose, mouth, and throat. Compositions for topical administration via the skin and mucous membranes should not give rise to signs of irritation, such as swelling or redness.

The topical composition may include a pharmaceutically acceptable carrier adapted for topical administration. Thus, the composition may take the form of a suspension, solution, ointment, lotion, cream, foam, aerosol, spray, suppository, implant, inhalant, tablet, capsule, dry powder, syrup, balm or lozenge, for example. Methods for preparing such compositions are well known in the pharmaceutical industry.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Compositions suitable for topical administration in the mouth include lozenges comprising active agents in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Creams, ointments or pastes according to the present invention are semi-solid compositions of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The composition may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

The pharmaceutical agent-chemical modifier complexes described herein can be administered transdermally. Transdermal administration typically involves the delivery of a pharmaceutical agent for percutaneous passage of the drug into the systemic circulation of the patient. The skin sites include anatomic regions for transdermally administering the drug and include the forearm, abdomen, chest, back, buttock, mastoidal area, and the like.

Transdermal delivery is accomplished by exposing a source of the complex to a patient's skin for an extended period of time. Transdermal patches have the added advantage of providing controlled delivery of a pharmaceutical agent-chemical modifier complex to the body. See Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Such dosage forms can be made by dissolving, dispersing, or otherwise incorporating the pharmaceutical agent-chemical modifier complex in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel.

A variety of types of transdermal patches will find use in the methods described herein. For example, a simple adhesive patch can be prepared from a backing material and an acrylate adhesive. The pharmaceutical agent-chemical modifier complex and any enhancer are formulated into the adhesive casting solution and allowed to mix thoroughly. The solution is cast directly onto the backing material and the casting solvent is evaporated in an oven, leaving an adhesive film. The release liner can be attached to complete the system.

Alternatively, a polyurethane matrix patch can be employed to deliver the pharmaceutical agent-chemical modifier complex. The layers of this patch comprise a backing, a polyurethane drug/enhancer matrix, a membrane, an adhesive, and a release liner. The polyurethane matrix is prepared using a room temperature curing polyurethane prepolymer. Addition of water, alcohol, and complex to the prepolymer results in the formation of a tacky firm elastomer that can be directly cast only the backing material

A further embodiment of this invention will utilize a hydrogel matrix patch. Typically, the hydrogel matrix will comprise alcohol, water, drug, and several hydrophilic polymers. This hydrogel matrix can be incorporated into a transdermal patch between the backing and the adhesive layer.

The liquid reservoir patch will also find use in the methods described herein. This patch comprises an impermeable or semipermeable, heat sealable backing material, a heat sealable membrane, an acrylate based pressure sensitive skin adhesive, and a siliconized release liner. The backing is heat sealed to the membrane to form a reservoir which can then be filled with a solution of the complex, enhancers, gelling agent, and other excipients.

Foam matrix patches are similar in design and components to the liquid reservoir system, except that the gelled pharmaceutical agent-chemical modifier solution is constrained in a thin foam layer, typically a polyurethane. This foam layer is situated between the backing and the membrane which have been heat sealed at the periphery of the patch.

For passive delivery systems, the rate of release is typically controlled by a membrane placed between the reservoir and the skin, by diffusion from a monolithic device, or by the skin itself serving as a rate-controlling barrier in the delivery system. See U.S. Pat. Nos. 4,816,258; 4,927,408; 4,904,475; 4,588,580, 4,788,062; and the like. The rate of drug delivery will be dependent, in part, upon the nature of the membrane. For example, the rate of drug delivery across membranes within the body is generally higher than across dermal barriers. The rate at which the complex is delivered from the device to the membrane is most advantageously controlled by the use of rate-limiting membranes which are placed between the reservoir and the skin. Assuming that the skin is sufficiently permeable to the complex (i.e., absorption through the skin is greater than the rate of passage through the membrane), the membrane will serve to control the dosage rate experienced by the patient.

Suitable permeable membrane materials may be selected based on the desired degree of permeability, the nature of the complex, and the mechanical considerations related to constructing the device. Exemplary permeable membrane materials include a wide variety of natural and synthetic polymers, such as polydimethylsiloxanes (silicone rubbers), ethylenevinylacetate copolymer (EVA), polyurethanes, polyurethane-polyether copolymers, polyethylenes, polyamides, polyvinylchlorides (PVC), polypropylenes, polycarbonates, polytetrafluoroethylenes (PTFE), cellulosic materials, e.g., cellulose triacetate and cellulose nitrate/acetate, and hydrogels, e.g., 2-hydroxyethylmethacrylate (HEMA).

Other items may be contained in the device, such as other conventional components of therapeutic products, depending upon the desired device characteristics. For example, the compositions according to this invention may also include one or more preservatives or bacteriostatic agents, e.g., methyl hydroxybenzoate, propyl hydroxybenzoate, chlorocresol, benzalkonium chlorides, and the like. These pharmaceutical compositions also can contain other active ingredients such as antimicrobial agents, particularly antibiotics, anesthetics, analgesics, and antipruritic agents.

### Compositions for Administration as Suppositories

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The active compound may be formulated into a suppository comprising, for example, about 0.5% to about 50% of a compound of the invention, disposed in a polyethylene glycol (PEG) carrier (e.g., PEG 1000 [96%] and PEG 4000 [4%].

### Administration

Any administration form that will ensure that the GLP-1 receptors will be exposed to sufficient levels of the bioactive form of a GLP-1 molecule may be part of the present invention. However, taken into consideration that the individuals to be treated possibly will have to receive treatment for a longer period, such as weeks or months, it is preferred that the administration form is well suited therefore.

Accordingly, it is preferred that the GLP-1 molecule is administered subcutaneously in an amount sufficient to allow sufficient levels of the bioactive form of ghrelin, i.e. the acylated form, to reach the receptors.

### Other Delivery Methods

In another embodiment, cell-based GLP-1 molecule delivery or gene therapy for long acting GLP-1 delivery may be used; these strategies would preferably employ endogenous and/or heterologous nutrient sensitive promoters for control of GLP-1 secretion.

In one preferred embodiment, a molecule may be administered that is capable of activating GLP-1 receptor signaling in target tissues and/or stimulating GLP-1 release from the endogenous enteroendocrine L cell.

### Medical Packaging

The compounds used in the invention may be administered alone or in combination with pharmaceutically acceptable carriers or excipients, in either single or multiple doses. The formulations may conveniently be presented in unit dosage form by methods known to those skilled in the art.

It is preferred that the compounds according to the invention are provided in a kit. Such a kit typically contains an active compound in dosage forms for administration. A dosage form contains a sufficient amount of active compound such that a desirable effect can be obtained when administered to a subject.

Thus, it is preferred that the medical packaging comprises an amount of dosage units corresponding to the relevant dosage regimen. Accordingly, in one embodiment, the medical packaging comprises a pharmaceutical composition comprising a compound as defined above or a pharmaceutically acceptable salt thereof and pharmaceutically acceptable carriers, vehicles and/or excipients, said packaging having from 7 to 21 dosage units, or multiples thereof, thereby having dosage units for one week of administration or several weeks of administration.

The dosage units can be as defined above. The medical packaging may be in any suitable form for parenteral, in particular subcutaneous administration. In a preferred embodiment the packaging is in the form of a cartridge, such as a cartridge for an injection pen, the injection pen being such as an injection pen known from insulin treatment.

When the medical packaging comprises more than one dosage unit, it is preferred that the medical packaging is provided with a mechanism to adjust each administration to one dosage unit only.

Preferably, a kit contains instructions indicating the use of the dosage form to achieve a desirable affect and the amount of dosage form to be taken over a specified time period. Accordingly, in one embodiment the medical packaging comprises instructions for administering the pharmaceutical composition.

### Combination Treatments

The GLP-1 molecules of the present invention may also be administered in combination with other agent(s). By administering “in combination” is meant that said other agents(s) may be administered prior to, simultaneously with (such as co-formulated with), or after administration of a GLP-1 molecule according to the present invention.

Thus, in one embodiment of the present invention, the GLP-1 molecule(s) may be administered in combination with one or more other excitatory factor(s) capable of inducing bile flow and/or treating biliary tract motility disorders, such as e.g. any of the following: Cholecystokinin (CCK), caerulein, acetylcholine, motilin, cholinomimetic drugs such as bethanecol or prostigmine, ursodeoxcholic acid, dietary fat, NSAIDS, erythromycin, cisapride, cholestyramine, i.v. administration of amino acids, and non-steroidal anti-inflammatory drugs

Further suitable agents to be administered in combination with the GLP-1 molecule in the present invention include, but are not restricted to:—
a systemic analgesic
hyoscine-N-butylbromide (Buscopan)
Pethidine (meperidine)
Nifedipine
isosorbide dinitrate
botulinum toxin (e.g. from Botox; Allergan, Inc., Irvine, Calif.)

In another embodiment of the present invention, the GLP-1 molecule(s) may be administered in combination with one or more inhibitory factor(s) capable of reducing bile flow, such as e.g. any of the following: said inhibitory factor is selected from any of the following factors: somatostatin, octreotide, Sandostatin LAR, anticholinerge drugs, Nitric oxide, L-Arginine, nitric oxide donors such as glyceryl trinitrate, nitroprusside, calcium chanel antagonists such as nifedipine and verapamil, loperamide, Nifedipine, progesterone, Trimebutine maleate, Loperamide or ondansetron.

In one preferred embodiment of the present invention, the GLP-1 molecule(s) may be administered in combination with one or more analgesic, such as e.g. any of the following: an opioid, an NSAID, an salicylic acid derivative, a paracetamol.

In another embodiment of the present invention, the GLP-1 molecule(s) may be administered in combination with one or more agents impacting biliary dyskinesia such as a nitrate or a calcium channel antagonist.

In one embodiment of the present invention, the GLP-1 molecule(s) may be administered in combination with one or more of the following:

### 1) Acarbose

### 2) Metformin

3) DPP-IV inhibitor Val-Pyr

In one preferred embodiment of the present invention, the GLP-1 molecule is administered in combination with one or more of the following: ioperamide, hyoscyamine sulphate, lactulose, YM-443, dexloxigiumide, bisacodyl, AZD 7371, tainetant (SB 223412), asimadoline, NT3, SLV305, AZD 3355, AZD 9343, pinaverine, Na picosulphate, calcium polycarpohil, trimebutine, dopamine D2 antagonists, such as itopride, motilin agonists, such as KC 11458 and GM-611, NK2 antagonists, such as saredutant, NK3/NK2 receptor antagonists, such as SSR 241586, 5-hydroxytryptamine₄ (5-HT₄) receptor partial agonists, such as tegaserod, 5-HT₄ receptor agonists, such as mosapride and prucalopride, antagonists of the serotonin 5-HT₃ receptor type, such as alosetron or cilansetron, dual 5-HT₄ agonist and 5-HT₃-antagonists, such as renzapride and E-3620, GLP-1 or analogues/homologues thereof, such as exenatide, exenatide LAR, liraglutide, CJC-1131, ZP 10/AVE-0010 and GTP-010, DPP-IV inhibitors, such as LAF237, PYY(1-36) or analogues/homologues thereof, PYY(3-36) or analogues/homologues thereof, NPY(1-36) or analogues/homologues thereof, NPY(3-36) or analogues/homologues thereof and anti-depressants, such as selective serotonin reuptake inhibitors (SSRIs), serotonin noradrenalin reuptake inhibitors (SNRIs), norepinephrine serotonin reuptake inhibitors (NSRIs), selective noradrenalin reuptake inhibitors, tetracyclic antidepressants, non-selective monoamine reuptake inhibitors including tricyclic antidepressants (TCAs), selective reversible monoamine reuptake inhibitors and antidepressants with other mechanisms of action, e.g. mirtazapin. Examples of SSRIs are citalopram, escitalopram, fluoxetine, fluvoxamine, paroxetine and sertraline. An example of an SNRI is venlafaxine. An example of an NSRI is milnacipran. A further example of an anti-depressant is duloxetine.

### EXAMPLES

### Example 1

### Suitable Formulation of a GLP-1 Molecule for Use in the Present Invention

Three formulations will be made as follows:

(A) A 21.5 ml aliquot of peptide solution in water will be mixed with 21.5 ml of 0.63% m cresol-3.2% glycerol and the final pH was set to 8.48. The solution will be passed through a 0.2 micron filter. Then aliquots of the solution, containing 0.5 mg/ml peptide in 0.315% m-cresol-1.6% glycerol at pH 8.48, will be pipetted into parenteral vials and stoppered.

(B) A 21.5 ml aliquot of peptide solution in water will be mixed with 21.5 ml of 0.63% m cresol-3.2% glycerol-0.02 molar L-Lysine pH 8.5 and the final pH will be set to 8.48.

The solution will be passed thru a 0.2 micron filter. Then, aliquots of the solution, containing 0.5 mg/ml peptide in 0.315% m-cresol-1.6% glycerol-0.01 molar L-Lysine at pH 8.48, will be pipetted into parenteral vials and stoppered.

(C) A 21.5 ml aliquot of peptide solution in water will be mixed with 21.5 ml of 0.63% m cresol-3.2% glycerol-0.02 molar Tris buffer pH 8.5 and the final pH will be set to 8.50.

The solution will be passed thru a 0.2 micron filter. Aliquots of the solution, containing 0.5 mg/ml peptide in 0.315% m-cresol-1.6% glycerol-0.01 molar tris at pH 8.50, will be pipetted into parenteral vials and stoppered.

Preparation of the GLP-1 Compounds for Use in the Present Invention Solid Phase t-Boc Chemistry

Approximately 0.5-0.6 grams (0.38-0.45 mmole) Boc Gly-PAM resin will be placed in a standard 60 ml reaction vessel and double couplings will be run on an Applied Biosystems ABI430A peptide synthesizer. The following side-chain protected amino acids (2 mmole cartridges of Boc amino acids) will be obtained from Midwest Biotech (Fishers, Ind.) and used in the synthesis:

Arg-Tosyl (TOS), Asp-.delta.-cyclohexyl ester(CHXL), Glu-.delta.-cyclohexyl ester (CHXL), His-benzyloxymethyl(BOM), Lys-2-chlorobenzyloxycarbonyl (2Cl-Z), Met-sulfoxide (O), Ser-O-benzyl ether (OBzl), Thr-O-benzyl ether (OBzl), Trp-formyl (CHO) and Tyr-2-bromobenzyloxycarbonyl (2Br-Z) and Boo Gly PAM resin. Trifiuoroacetic acid (TFA), di-isopropylethylamine (DIEA), 0.5 M hydroxybenzotriazole (HOBt) in DMF and 0.5 M dicyclohexylcarbodiimide (DCC) in dichloromethane were purchased from PE-Applied Biosystems (Foster City, Calif.). Dimethylformamide (DMF-Burdick and Jackson) and dichloromethane (DCM-Mallinkrodt) were purchased from Mays Chemical Co. (Indianapolis, Ind.).

Standard double couplings will be run using either symmetric anhydride or HOBt esters, both formed using DCC. A second set of double couplings (without TFA deprotection) will be run at Trp31, Thr13 and Thr11. At the completion of the syntheses, the N-terminal Boc group will be removed and the peptidyl resins treated with 20% piperidine in DMF to deformylate the Trp side chain. After washing with DCM, the resins will be transferred to a TEFLON reaction vessel and dried in vacuo.

For analogs containing Met, an on-the-resin reduction will be done using TFA/10% dimethyl sulfide (DMS)/2% concentrated HCl. Cleavages will be done by attaching the reaction vessels to a HF (hydrofluoric acid) apparatus (Penninsula Laboratories). 1 ml m-cresol per gram/resin will be added and 10 ml HF (purchased from AGA, Indianapolis, Ind.) will be condensed into the pre-cooled vessel. 1 ml DMS per gram resin will be s added when methionine is present. The reactions will be stirred one hour in an ice bath and the HF removed in vacuo. The residues will be suspended in ethyl ether and the solids will be filtered and washed with ether. Each peptide will be extracted into aqueous acetic acid and either freeze dried or loaded directly onto a reverse-phase column.

Purifications will be run on a 2.2.times.25 cm VYDAC C18 column in buffer A (0.1% Trifluoroacteic acid in water, B: 0.1% TFA in acetonitrile). A gradient of 20% to 90% B will be run on an HPLC (Waters) over 120 minutes at 10 ml/minute while monitoring the UV at 280 nm (4.0 A) and collecting one minute fractions.

Appropriate fractions will be combined, frozen and lyophilized. Dried products will be analyzed by HPLC (0.46.times.15 cm METASIL AQ C18) and MALDI mass spectrometry.

### Preparation of the GLP-1 Compounds of the Present Invention by Solid Phase F-Moc Chemistry

Approximately 114 mg (50 mMole) FMOC Gly WANG resin (purchased from NovaBiochem, LaJolla, Calif.) will be placed in each programmed well of the 96 well reaction block and double couplings will be run on an Advanced ChemTech 396 peptide synthesizer. Analogs with a C-terminal amide will be prepared using 75 mg (50.mu.mole) Rink Amide AM resin (NovaBiochem, LaJolia, Calif.).

The following FMOC amino acids will be purchased from Advanced ChemTech (Louisville, Ky.), NovaBiochem (La Jolla, Calif.), and Midwest BioTech (Fishers, Ind.): Arg-2,2,4,6,7-pentamethyldihydrobenzofuran-5-sul-fonyl (Pbf), Asn-trityl (Trt), Asp-.beta.-t-Butyl ester (tBu), Glu-.delta.-t-butyl ester (tBu), Gln-trityl (Trt), His-trityl (Trt), Lys-t-butyloxycarbonyl (Boc), Ser-t-butyl ether (OtBu), Thr-t-butyl ether (OtBu), Trp-t-butyloxycarbonyl (Boc), Tyr-t-butyl ether (OtBu).

Solvents dimethylformamide (DMF-Burdick and Jackson), N-methylpyrrolidone

(NMP-Burdick and Jackson), dichloromethane (DCM-Mallinkrodt) will be purchased from Mays Chemical Co. (Indianapolis, Ind.).

Hydroxybenzotrizole (HOBt), di-isopropylcarbodiimde (DIC), di-isopropylethylamine (DIEA), and piperidine (Pip) will be purchased from Aldrich Chemical Co (Milwaukee, Wis.).

All amino acids will be dissolved in 0.45 M HOBt in NMP and 50 minutes DIC/IHOBt activated couplings will be run after 20 minutes deprotection using 20% Pip/DMF. Each resin will be washed with DMF after deprotections and couplings. After the last coupling and deprotection, the peptidyl resins will be washed with DCM and dried in vacuo in the reaction block.

With the reaction/cleavage block assembly in place, 2 ml Reagent K will be added to each well and the cleavage reaction mixed for 2 hours [Reagent K=0.75 gm phenol, 0.5 ml thioanisole, 0.25 ml ethanedithiol, 0.5 ml water per 10 ml trifluoroacetic acid (TFA), all can be purchased from Aldrich Chemical Co., Milwaukee, Wis.]. The TFA filtrates will be added to 40 ml ethyl ether and the precipitants centrifuged 2 minutes at 2000 rpm. The supernatants will be decanted, the pellets re-suspended in 40 ml ether, re-centrifuged, re-decanted, dried under nitrogen and then in vacuo.

0.3-0.6 mg of each product will be dissolved in 1 ml 0.1% TFA/acetonitrile(ACN) and 20 ul was analyzed on HPLC (0.46.times.15 cm METASIL AQ C18, 1 ml/min, 45.degree. C., 214 nM (0.2 A), A=0.1% TFA, B=0.1% TFA/50% ACN. Gradient=50% B to 90% B over 30 minutes].

Purifications will be run on a 2.2.times.25 cm VYDAC C18 column in buffer A (0.1% trifluoroacteic acid in water, B: 0.1% TFA in acetonitrile). A gradient of 20% to 90% B will be run on an HPLC (Waters) over 120 minutes at 10 ml/minute while monitoring the UV at 280 nm (4.0 A) and collecting 1 minute fractions. Appropriate fractions will be combined, frozen and lyophilized. Dried products will be analyzed by HPLC (0.46.times.15 cm METASIL AQ C18) and MALDI mass spectrometry.

### Example 2

### Screening Method for Evaluating Effect of an Agent on Motility of a Component of the Binary Tract

Overview: Guinea pigs will be killed to remove the whole gallbladder. Two or three smooth muscle strips (8 mm×3 mm) will be cut along the longitudinal direction. The mucosa on each strip will be carefully removed. Each longitudinal muscle strip will be suspended in a tissue chamber containing 5 mL Krebs solution (37° C.), bubbled continuously with 950 mL/L O₂ and 50 mL/L CO₂. The resting tension (g), mean contractile amplitude (mm), and contractile frequency (waves/min) will be simultaneously recorded on recorders. After 2 h equilibration, the agent to be evaluated for effect on motility of the smooth muscle will be added cumulatively to the tissue chamber in turns every 2 min to observe the effect on the gallbladder.

### Animal Preparation

Guinea pigs of either sex (grade I, e.g. purchased from Animal Center of Lanzhou Biology Institute), weighing 350-450 g, will be fasted with free access to water for 24 h, and killed to remove the whole gallbladder. Two or three smooth muscle strips (8 mm×3 mm) will be cut along the longitudinal direction. The mucosa on each strip will be carefully removed.

### Experiment

The muscle strips will be suspended in a tissue chamber containing 5 mL Krebs solution, constantly warmed by a circulating water jacket at 37° C., bubbled continuously with 950 mL/L O₂ and 50 mL/L CO₂. One end of the strip will be fixed to a hook on the bottom of the chamber. The other end will be connected to an external isometric force transducer (JZ-BK, BK). The preparation will be subjected to 1-g-load tension and washed with 5 mL Krebs solution every 20 min. The motility of gallbladder strips in tissue chambers will be simultaneously recorded on ink writing two channel recorders (LMS-ZB, Chengdu). After 2-h equilibration, the agent to be evaluated will be added cumulatively in turns every 2 min to observe their effects on gallbladder.

The results will be presented as mean±SE, and statistically analyzed by ANOVA, P<0.05 to be considered statistically significant.

It is to be understood that this protocol may also be adapted to measure motility of other areas of the biliary system.

1. A method for the treatment or prevention of biliary dyskinesia and/or biliary pain or discomfort which comprises administration of a therapeutically or prophylactically effective amount of a GLP-1 molecule to an individual in need thereof.

2. The method according to claim 1, wherein said GLP-1 molecule is defined as one or more of the following:
a) comprising an amino acid sequence capable of being recognized by an antibody, said antibody also recognizing human GLP-1, and/or
b) comprising an amino acid sequence capable of binding selectively to the human GLP-1 receptor, or
c) a small molecule capable of binding selectively to the human GLP-1 receptor.

3. The method according to claim 1, wherein said GLP-1 molecule is GLP-1 (7-36)amide or GLP-1 (7-37), or a fragment, analog, derivative or homologue thereof.

4. The are method according to claim 3, wherein said homologue is at least 80% homologous to GLP-1 (7-36)amide or GLP-1 (7-37).

5. The method according to claim 3, wherein said homologue is at least 90% homologous to GLP-1 (7-36)amide or GLP-1 (7-37).

6. The method according to claim 3, wherein said homologue is at least 95% homologous to GLP-1 (7-36)amide or GLP-1 (7-37).

7. The method according to claim 1, wherein the GLP-1 molecule comprises fewer than 20 non-natural amino acid residues or D-amino acids.

8. The method according to claim 1, wherein said GLP-1 molecule comprises a C-terminal functional group modification and/or an N-terminal functional group modification.

9. The method according to claim 1, wherein said GLP-1 molecule is selected from any of the following: GLP-1 (7-36)amide, GLP-1 (7-37), exendin-4, GLP-1 (7-34), GLP-1 (7-35), GLP-1 (7-36), Gln9-GLP-1 (7-37), D-Gln9-GLP-1 (7-37), Thr16-Lys18-GLP-1 (7-37), Lys18-GLP-1 (7-37), GLP-1 (7-34), GLP-1 (7-35), GLP-1 (7-36), GLP-1 (7-36)NH2, Gly8-GLP-1 (7-36)NH2, Gln9-GLP-1 (7-37), D-Gln9-GLP-1 (7-37), acetyl-Lys9-GLP-1 (7-37), Thr9-GLP-1 (7-37), D-Thr9-GLP-1 (7-37), Asn9-GLP-1 (7-37), D-Asn9-GLP-1 (7-37), Ser22-Arg23-Arg24-Gln26-GLP-1 (7-37), Thr16-Lys18-GLP-1 (7-37), Lys18-GLP-1 (7-37), Arg23-GLP-1 (7-37), Arg24-GLP-1 (7-37), GLP-1 (7-34), GLP-1 (7-35), GLP-1 (7-36) NH2, Gln9-GLP-1 (7-37), d-Gln9-GLP1 (7-37), Thr16-Lys18-GLP-1 (7-37), Lys18-GLP-1 (7-37), Gly′-GLP-1 (7-36) NH2, Gly′-GLP1 (7-37) OH, Val′-GLP-1 (7-37) OH, Met8-GLP-1 (7-37) OH, acetyl-Lys9-GLP-1 (7-37), Thr9 GLP-1 (7-37), D-Thr9-GLP-1 (7-37), Asn9-GLP-1 (7-37), D-Asn9-GLP-1 (7-37), Ser22-Arg23 Arg24-Gln26-GLP-1 (7-37), Arg23-GLP-1 (7-37), Arg24-GLP-1 (7-37), a-methyl-Ala8-GLP-1 (736) NH2, Gly′-Gln2′-GLP-1 (7-37) OH or LY315902.

10. The method according to claim 1, wherein said GLP-1 molecule is selected from GLP-1 (7-36)amide, GLP-1 (7-37), Liraglutide, Exanatide, Albugon, CJC-1131, zp-10, BIM51077 (Ipsen), LY315902, LY307161 (Eli Lilly), S 23521.

11. The method according to claim 1, wherein the individual is a human being.

12. The method according to claim 1, wherein the biliary dyskinesia and/or biliary pain is associated with a condition selected from the group consisting of: gallstones, inflammatory bowel disease, chronic acalculous cholecystitis, alcalculous gallbladder disease, cystic duct syndrome, acalculous gallbladder disease, Treatment with gall-bladder motility inhibiting drugs, parenteral nutrition, obesity, diabetes mellitus, coeliac disease, diabetic neuropathy, Downs syndrome, Beta-thalassemia, Pregnancy, gastric surgery, very low calorie dieting, extracorporeal shockwave lithotripsy, Somatostatin-producing tumour, sphincter of Oddi stenosis, sphincter of Oddi dyskinesia, glandular hyperplasia, SO muscular incoordination or muscular hypertonicity (spasm), Hyperthyroidism, Treatment with a compound known to impair the passage of bile Biliary tract obstruction (partial or complete) Cholangiocarcinoma, Primary sclerosing cholangitis (PSC), Tumors of the ampulla of Vater, Mirizzi's syndrome, other biliary tumour types, AIDS cholangiopathy, infections, Obstructive lesion in the large ducts of the biliary tree outside the liver, primary biliary cirrhosis, primary sclerosing cholangitis, infantile obstructive cholangiopathy, damage to the small intraheptic bile ducts by drugs biliary sludge or and cholestasis.

13. The method according to claim 1, wherein said pain and/or dyskinesia is in one or more of the following locations of the biliary tract: gallbladder, sphincter of Oddi, bile duct wall and/or cystic duct.

14. The method according to claim 13, wherein said dyskinesia and/or pain is in the gallbladder.

15. The method according to claim 14, wherein said individual is suffering from, or at risk of suffering from a pathological condition selected from the group consisting of gallstones, inflammatory bowel disease, chronic acalculous cholecystitis, alcalculous gallbladder disease, cystic duct syndrome and acalculous gallbladder disease.

16. The method according to claim 14, wherein said individual is suffering from, or at risk of suffering from a condition selected from the group consisting of Treatment with gall-bladder motility inhibiting drugs, parenteral nutrition, obesity, diabetes mellitus, coeliac disease, diabetic neuropathy, Downs syndrome, Beta-thalassemia, Pregnancy, gastric surgery, very low calorie dieting, extracorporeal shockwave lithotripsy and Somatostatin-producing tumour.

17. The method according to claim 13, wherein said dyskinesia and/or pain is in the sphincter of Oddi.

18. The method according to claim 17, wherein said individual is suffering from, or at risk of suffering from, any of the following pathological conditions: “sphincter of Oddi stenosis”, sphincter of Oddi dyskinesia, or hyperthyroidism.

19. The method according to claim 1, wherein said individual has been, is being or is going to be treated with an inhibitory factor (such as a drug) known to inhibit biliary tract motility and/or reduce the basal tone of the biliary system.

20. The method according to claim 19, wherein said inhibitory factor is selected from the group of factors consisting of: somatostatin, octreotide, Sandostatin LAR, anticholinergic drugs, Nitric oxide, L-Arginine, nitric oxide donors, nitroprusside, calcium channel antagonists, loperamide, Nifedipine, progesterone, Trimebutine maleate, Loperamide and ondansetron.

21. The method according to claim 1, wherein said individual is suffering from, or at risk of suffering from, a pathological condition selected from the group consisting of Biliary tract obstruction (partial or complete), Cholangiocarcinoma, Primary sclerosing cholangitis (PSC), Tumors of the ampulla of Vater, Other biliary tumour types, Mirizzi's syndrome, AIDS cholangiopathy, an infection, Obstructive lesion in the large ducts of the biliary tree outside the liver, primary biliary cirrhosis, primary sclerosing cholangitis, infantile obstructive cholangiopathy, extrahepatic biliary atresia, drug damage to the small intraheptic bile ducts, biliary sludge, and cholestasis.

22. The method according to claim 1, wherein the GLP-1 molecule is administered in an amount of from 1 μg/kg to about 100 mg/kg per day.

23. The method according to claim 22, wherein the GLP-1 molecule is administered in a dosage of 0. 4-2. 4 pmol kg⁻¹ min⁻¹.

24. The method according to claim 23, wherein the GLP-1 molecule is administered in a dosage of 150-350 ug/kg, preferably daily.

25. The method according to claim 1, wherein the GLP-1 molecule is administered orally, parenterally, intravenously, subcutaneously, or via the intranasal, buccal or sublingual routes.

26. An in vitro method for evaluating the capability of an agent to modulate the motility of at least one area of the biliary system in a mammal in need thereof, said method comprising the steps of:
(a) providing an assay for measuring the binding of said agent to the GLP-1 receptor
(b) adding the agent to be tested to the assay, and
(c) determining the amount of said compound bound to the GLP-1 receptor, and
(d) optionally comparing the amount determined in step c) with an amount measured in the absence of the agent to be tested,
wherein a difference in the two amounts identifies an agent capable of modulating the GLP-1 receptor.

27. The method of claim 26 comprising the additional step of refining the isolated compound and/or reducing the toxicity of the isolated compound.

28. The method according to claim 27, comprising the further step of formulating the refined compound/compound with reduced toxicity with a pharmaceutically acceptable carrier or diluent.

29. A combination of a GLP-1 molecule and an analgesic.

30. (canceled)

31. The combination of claim 29 wherein the analgesic is selected from the group consisting of an opioid, an NSAID, an salicylic acid derivative and a paracetamol.
